(19) United States
(12) Patent Application Publication
(10) Pub. No.: US 2025/0163065 A1
(43) Pub. Date: May 22, 2025

(54) PYRROLOTRIAZINONE COMPOUND, PHARMACEUTICAL COMPOSITION COMPRISING SAME, PREPARATION METHOD THEREFOR, AND USE THEREOF

(71) Applicant: SHANGHAITECH UNIVERSITY, Shanghai (CN)

(72) Inventors: Jianjun CHENG, Shanghai (CN); Zhijie LIU, Shanghai (CN); Tian HUA, Shanghai (CN); Jianhang MAO, Shanghai (CN); Ning ZHOU, Shanghai (CN)

(21) Appl. No.: 18/840,522

(22) PCT Filed: Jan. 6, 2023
(86) PCT No.: PCT/CN2023/071042

§ 371 Date: Dec. 10, 2024

(30) Foreign Application Priority Data
Mar. 1, 2022 (CN) 202210195902.X

(51) Int. Cl. C07D 487/04; A61K 31/53; A61K 31/5377; A61P 25/18

(52) U.S. Cl. CPC C07D 487/04; A61K 31/53; A61K 31/5377; A61P 25/18

(57) ABSTRACT

Provided are a pyrrolotriazinone compound represented by formula I or a pharmaceutically acceptable salt thereof, a pharmaceutical composition comprising same, a preparation method therefor, and use thereof. The pyrrolotriazinone compound has agonistic activity on GPR139 receptor.

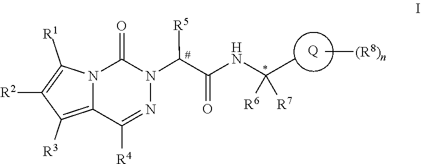

The present application claims the right of priority of Chinese patent application No. 202210195902X filed on Mar. 1, 2022. The contents of the above Chinese patent application are incorporated herein by reference in its entirety.

### TECHNICAL FIELD

The present disclosure relates to a pyrrolotriazinone compound, a pharmaceutical composition comprising same, a preparation method therefor, and a use thereof.

### BACKGROUND

Schizophrenia affects more than 20 million people worldwide. Its symptoms can be roughly divided into three categories based on their clinical characteristics: positive symptoms, negative symptoms, and cognitive dysfunction. Positive symptoms refer to exaggerations or distortions of normal functional behaviors, including hallucinations, delusions, confusion, etc. Negative symptoms refer to some defects in normal emotional responses or other thought processes, including flat affect, alogia, loss of motivation and pleasure, and depressive-like features such as a negative attitude and social withdrawal. Cognitive dysfunction mainly manifests as decline in attention, memory, learning ability, etc.

Based on the historical context of drug development and pharmacological properties, current anti-schizophrenia drugs can be divided into three generations. First-generation anti-schizophrenia drugs, also known as typical anti-schizophrenia drugs, are potent antagonists of dopamine D2 receptors. Currently commonly used first-generation anti-schizophrenia drugs include haloperidol, chlorpromazine, perphenazine, etc., which can effectively suppress the positive symptoms of schizophrenia, but long-term use of these drugs can induce extrapyramidal reactions similar to Parkinson's disease. Second-generation anti-schizophrenia drugs, including clozapine, risperidone, and paliperidone, exert their effects by dual blockade of dopamine D2 receptors and serotonin 2A (5-HT_{2A}) receptors. Since inhibition of 5-HT_{2A} receptors can indirectly promote the release of dopamine from dopaminergic neurons, the extrapyramidal side effects induced by second-generation drugs are significantly reduced. However, the risk of metabolic syndrome (excessive obesity, insulin resistance, dyslipidemia, and hypertension) is higher with second-generation drugs than with typical anti-schizophrenia drugs. The third-generation anti-schizophrenia drugs include aripiprazole, brexpiprazole, and cariprazine, which are partial agonists of dopamine D2 receptors. As a “stabilizer” for dopamine, the third-generation drugs stabilize the activity of the brain's dopamine circuit in a moderate range, greatly reducing extrapyramidal side effects and significantly improving safety. The second and third generations are also collectively known as atypical anti-schizophrenia drugs. Currently, all drugs for the treatment of schizophrenia can effectively alleviate positive symptoms, but most drugs have limited or ineffective efficacy in improving negative symptoms and cognitive function. Therefore, there is an urgent need to develop drugs with novel mechanisms of action targeting the negative symptoms and cognitive disorder associated with schizophrenia.

The orphan receptor GPR139 was identified from bioinformatics analysis of the human genome and belongs to class A G protein-coupled receptors (GPCRs). In mammals, GPR139 is mainly expressed in the central nervous system, with the highest expression sites being the striatum, pituitary, habenula, thalamus, and hypothalamus (Matsuo et al., Biochem Biophys Res Commun 2005, 331:363-369). In both rodents and humans, GPR139 is highly expressed in the medial habenula. Several studies have shown a link between the habenula and schizophrenia. Overall structural damage to the rodent habenula can lead to schizophrenia-related symptoms such as reduced social activities, cognitive impairment, and excessive responses to stressful stimuli (Wang et al., Neuroreport 2013, 24:276-280). Patients with chronic schizophrenia have a higher frequency of habenula calcification and changes in habenula volume than normal individuals (Sandyk et al., Int J Neurosci 1992, 67:19-30). Functional magnetic resonance imaging (fMRI) studies have shown that, during a matching task, the habenula is activated in normal individuals when an error occurs, but in patients with chronic schizophrenia, their habenula is not significantly activated after a matching error occurs (Shepard et al., Schizophr Bull 2006, 32:417-421). The GPR139 knockout mouse model shows behavioral characteristics associated with schizophrenia such as decreased spontaneous movement, sensorimotor gating defects, and cognitive disorder. Symptoms can be improved by administering the dopamine D2 receptor antagonist haloperidol (Dao et al., Neuropsychopharmacology 2021). Based on these results, targeting GPR139 has the potential to develop new anti-schizophrenia drugs, especially drugs targeting negative symptoms.

The selective GPR139 agonist JNJ-63533054 (EC₅₀=16 nM) reported in 2018 can reduce self-drinking and hyperalgesia in alcohol-dependent rats (Kononoff et al., eNeuro 2018, 5), but did not show obvious behavioral modulation in in vivo studies. In 2021, Takeda Company of Japan announced TAK-041, a selective GPR139 agonist with a benzotriazinone structure (EC₅₀=22 nM), which has been confirmed to be effective in treating negative symptoms associated with schizophrenia in a mouse model (Reichard et al., J Med Chem 2021, 64:11527-11542) and has successfully entered Phase II clinical trials.

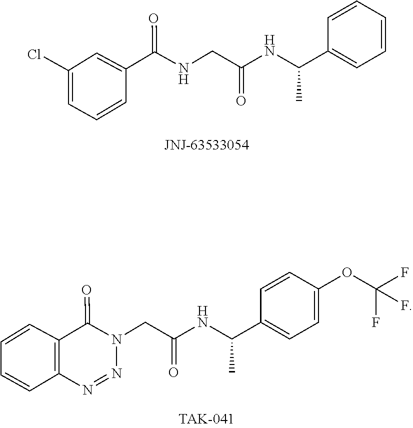 [SMILES: C[C@H](NC(=O)CNC(=O)c1cccc(Cl)c1)c1ccccc1 ; C[C@H](NC(=O)Cn1nnc2ccccc2c1=O)c1ccc(OC(F)(F)F)cc1]

### CONTENT OF THE PRESENT INVENTION

The technical problem to be solved by the present disclosure is the defect of the existing GPR139 receptor agonists with a single structure, and the present disclosure provides a pyrrolotriazinone compound, a pharmaceutical composition comprising same, a preparation method therefor, and a use thereof. The pyrrolotriazinone compound of the present disclosure has a novel structure and has strong agonistic activity on GPR139 receptors.

The present disclosure solves the above technical problem through the following technical solutions.

The present disclosure provides a pyrrolotriazinone compound of formula I or a pharmaceutically acceptable salt thereof;

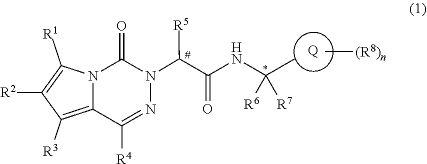

wherein
R¹, R², R³, and R⁴ are independently H, halogen, C₁₋₆ alkyl, C₃₋₆ cycloalkyl, —O—C₁₋₆ alkyl, C₁₋₆ alkyl substituted by 1, 2, or 3 R¹⁻¹, C₃₋₆ cycloalkyl substituted by 1, 2, or 3 R¹⁻², or —O—C₁₋₆ alkyl substituted by 1, 2, or 3 R¹⁻³;
R¹⁻¹, R¹⁻², and R¹⁻³ are independently halogen;
R⁵ is H or C₁₋₆ alkyl;
R⁶ and R⁷ are independently H, C₁₋₆ alkyl, C₃₋₆ cycloalkyl, C₁₋₆ alkyl substituted by 1, 2, or 3 R⁶⁻¹, or C₃₋₆ cycloalkyl substituted by 1, 2, or 3 R⁶⁻²;
R⁶⁻¹ is independently halogen, —O—C₁₋₆ alkyl, or —NR^{a}R^{b};
R⁶⁻² is independently halogen, —O—C₁₋₆ alkyl, or —NR^{a}R^{b};
R^{a} and R^{b} are independently C₁₋₆ alkyl;
or, R^{a} and R^{b}, together with the nitrogen atom to which they are attached, form a 5- to 6-membered heterocycloalkyl group or a 5- to 6-membered heterocycloalkyl group substituted by 1, 2, or 3 R^{a-1}; the 5- to 6-membered heterocycloalkyl group is independently a 5- to 6-membered heterocycloalkyl group with 1, 2, or 3 heteroatoms independently selected from 1, 2, or 3 kinds of N, O, and S;
R^{a-1} is independently C₁₋₆ alkyl;
Q is a C₆₋₁₀ aromatic ring, a 5- to 10-membered heteroaromatic ring, C₃₋₆ cycloalkyl, or 5- to 6-membered heterocycloalkyl; the 5- to 10-membered heteroaromatic ring is a 5- to 10-membered heteroaromatic ring with 1, 2, or 3 heteroatoms independently selected from 1, 2, or 3 kinds of N, O, and S;
n is 0, 1, 2, 3, 4, or 5;
R⁸ is independently halogen, —OH, —CN, —NH₂, C₁₋₆ alkyl, —O—C₁₋₆ alkyl, C₁₋₆ alkyl substituted by 1, 2, or 3 R⁸⁻¹, or —O—C₁₋₆ alkyl substituted by 1, 2, or 3 R⁸⁻²;
R⁸⁻¹ and R⁸⁻² are independently halogen;
or, R⁸ and R⁷ together form —(CH₂)ₘ—, m is 2 or 3;
carbon atoms with “*” and “#” are either achiral carbon atoms or chiral carbon atoms, and when they are chiral carbon atoms, they are independently in S configuration and/or R configuration.

In a certain embodiment, in the pyrrolotriazinone compound of formula I or the pharmaceutically acceptable salt thereof, certain groups can be defined as described below, and other groups can be defined as described in any embodiment of the present disclosure (hereinafter referred to as “in a certain embodiment”):

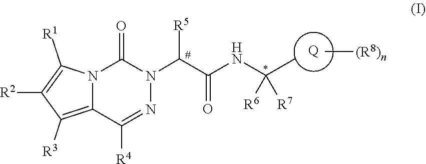

wherein
R¹, R², R³, and R⁴ are independently H, halogen, C₁₋₆ alkyl, C₃₋₆ cycloalkyl, —O—C₁₋₆ alkyl, C₁₋₆ alkyl substituted by 1, 2, or 3 R¹⁻¹, C₃₋₆ cycloalkyl substituted by 1, 2, or 3 R¹⁻², or —O—C₁₋₆ alkyl substituted by 1, 2, or 3 R¹⁻³;
R¹⁻¹, R¹⁻², and R¹⁻³ are independently halogen;
R⁵ is H or C₁₋₆ alkyl;
R⁶ and R⁷ are independently H, C₁₋₆ alkyl, C₃₋₆ cycloalkyl, C₁₋₆ alkyl substituted by 1, 2, or 3 R⁶⁻¹, or C₃₋₆ cycloalkyl substituted by 1, 2, or 3 R⁶⁻²;
R⁶⁻¹ is independently halogen, —O—C₁₋₆ alkyl, or —NR^{a}R^{b};
R⁶⁻² is independently halogen, —O—C₁₋₆ alkyl, or —NR^{a}R^{b};
R^{a} and R^{b} are independently C₁₋₆ alkyl;
or, R^{a} and R^{b}, together with the nitrogen atom to which they are attached, form a 5- to 6-membered heterocycloalkyl group or a 5- to 6-membered heterocycloalkyl group substituted by 1, 2, or 3 R^{a-1}; the 5- to 6-membered heterocycloalkyl group is independently a 5- to 6-membered heterocycloalkyl group with 1, 2, or 3 heteroatoms independently selected from 1, 2, or 3 kinds of N, O, and S;
R^{a-1} is independently C₁₋₆ alkyl;
Q is a C₆₋₁₀ aromatic ring or a 5- to 10-membered heteroaromatic ring; the 5- to 10-membered heteroaromatic ring is a 5- to 10-membered heteroaromatic ring with 1, 2, or 3 heteroatoms independently selected from 1, 2, or 3 kinds of N, O, and S;
n is 0, 1, 2, 3, 4, or 5;
R⁸ is independently halogen, —OH, —CN, —NH₂, C₁₋₆ alkyl, —O—C₁₋₆ alkyl, C₁₋₆ alkyl substituted by 1, 2, or 3 R⁸⁻¹, or —O—C₁₋₆ alkyl substituted by 1, 2, or 3 R⁸⁻²;
R⁸⁻¹ and R⁸⁻² are independently halogen;
or, R⁸ and R⁷ together form —(CH₂)ₘ—, m is 2 or 3;
carbon atoms with “*” and “#” are either achiral carbon atoms or chiral carbon atoms, and when they are chiral carbon atoms, they are independently in S configuration and/or R configuration.

In a certain embodiment,

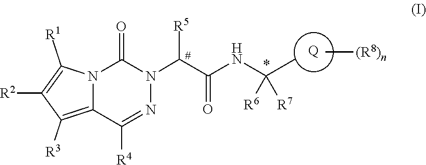

is

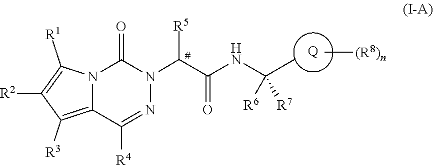

or

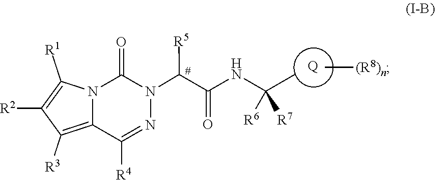

for example,

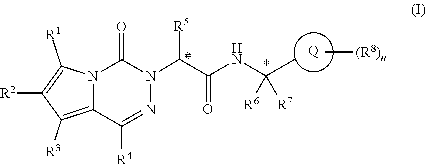

is

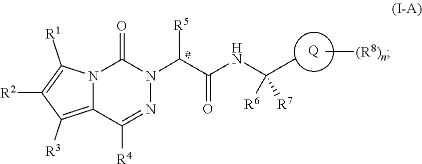

wherein *, #, R¹, R², R³, R⁴, R⁵, R⁶, R⁷, R⁸, Q, and n are defined as above.

In a certain embodiment, R¹, R², and R³ are independently H or C₁₋₆ alkyl.

In a certain embodiment, R⁴ is independently H.

In a certain embodiment, R⁵ is H.

In a certain embodiment, R⁶ is H.

In a certain embodiment, R⁷ is C₁₋₆ alkyl or C₁₋₆ alkyl substituted by 1, 2, or 3 R⁶⁻¹.

In a certain embodiment, R⁶⁻¹ is independently —NR^{a}R^{b}.

In a certain embodiment, R^{a} and R^{b} are independently C₁₋₆ alkyl. In a certain embodiment, n is 0 or 1.

In a certain embodiment, R⁸ is halogen, —OH, —CN, C₁₋₆ alkyl, —O—C₁₋₆ alkyl, C₁₋₆ alkyl substituted by 1, 2, or 3 R⁸⁻¹, or —O—C₁₋₆ alkyl substituted by 1, 2, or 3 R⁸⁻²;
or, R⁸ and R⁷ together form —(CH₂)ₘ—, m is 2 or 3; for example, R⁸ is halogen, —OH, C₁₋₆ alkyl, —O—C₁₋₆ alkyl, or —O—C₁₋₆ alkyl substituted by 1, 2, or 3 R⁸⁻²; or, R⁸ and R⁷ together form —(CH₂)ₘ—, m is 2 or 3.

In a certain embodiment, in R¹, R², R³, and R⁴, the C₁₋₆ alkyl is independently methyl, ethyl, n-propyl, isopropyl, n-butyl, isobutyl, or tert-butyl, for example, methyl.

In a certain embodiment, in R¹⁻¹, R¹⁻², and R¹⁻³, the halogen is F, Cl, Br, or I.

In a certain embodiment, in R⁶ and R⁷, the “C₁₋₆ alkyl” in the C₁₋₆ alkyl and C₁₋₆ alkyl substituted by 1, 2, or 3 R⁶⁻¹ is independently methyl, ethyl, n-propyl, isopropyl, n-butyl, isobutyl, or tert-butyl, for example, methyl or ethyl.

In a certain embodiment, in R^{a} and R^{b}, the C₁₋₆ alkyl is independently methyl, ethyl, n-propyl, isopropyl, n-butyl, isobutyl, or tert-butyl, for example, methyl.

In a certain embodiment, when R^{a} and R^{b}, together with the nitrogen atom to which they are attached, form a 5- to 6-membered heterocycloalkyl group or a 5- to 6-membered heterocycloalkyl group substituted by 1, 2, or 3 R^{a-1}, the “5- to 6-membered heterocycloalkyl group” in the 5- to 6-membered heterocycloalkyl group or 5- to 6-membered heterocycloalkyl group substituted by 1, 2, or 3 R^{a-1} is independently a 5- to 6-membered heterocycloalkyl group with 1 or 2 heteroatoms independently selected from N and O; for example,

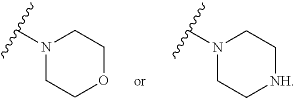 [SMILES: *N1CCOCC1 ; *N1CCNCC1]

In a certain embodiment, in R^{a-1}, the C₁₋₆ alkyl is independently methyl, ethyl, n-propyl, isopropyl, n-butyl, isobutyl, or tert-butyl, for example, methyl.

In a certain embodiment, in Q, the C₆₋₁₀ aromatic ring is a benzene ring or a naphthalene ring, for example, a benzene ring.

In a certain embodiment, in Q, the 5- to 10-membered heteroaromatic ring is a 5- to 6-membered heteroaromatic ring, for example, a pyridine ring, for another example,

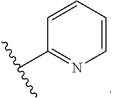 [SMILES: *c1ccccn1]

In a certain embodiment, in Q, the C₃₋₆ cycloalkyl is cyclopropyl, cyclobutyl, cyclopentyl, or cyclohexyl, for example, cyclohexyl.

In a certain embodiment, in R⁸, the halogen is independently F, Cl, Br, or I, for example F, Cl, or Br.

In a certain embodiment, in R⁸, the C₁₋₆ alkyl is independently methyl, ethyl, n-propyl, isopropyl, n-butyl, isobutyl, or tert-butyl, for example, methyl.

In a certain embodiment, in R⁸, the “—O—C₁₋₆ alkyl” in the —O—C₁₋₆ alkyl and —O—C₁₋₆ alkyl substituted by 1, 2, or 3 R⁸⁻² is independently methoxy, ethoxy, n-propoxy, isopropoxy, n-butoxy, isobutoxy, or tert-butoxy, for example, methoxy.

In a certain embodiment, in R⁸⁻¹ and R⁸⁻², the halogen is independently F, Cl, Br, or I, for example, F.

In a certain embodiment, when R⁸ and R⁷ together form —(CH₂)ₘ—, m is 3.

In a certain embodiment, when Q is a benzene ring, then

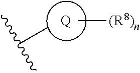

is

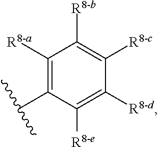

R^{8-a}, R^{8-b}, R^{8-c}, and R^{8-d} are independently H, halogen, —OH, —CN, —NH₂, C₁₋₆ alkyl, —O—C₁₋₆ alkyl, C₁₋₆ alkyl substituted by 1, 2, or 3 R⁸⁻¹, or —O—C₁₋₆ alkyl substituted by 1, 2, or 3 R⁸⁻²;
R^{8-e} is H, halogen, —OH, —CN, —NH₂, C₁₋₆ alkyl, —O—C₁₋₆ alkyl, C₁₋₆ alkyl substituted by 1, 2, or 3 R⁸⁻¹, or —O—C₁₋₆ alkyl substituted by 1, 2, or 3 R⁸⁻²;
or, R^{8-e} and R⁷ together form —(CH₂)ₘ—, m is 2 or 3.

In a certain embodiment,

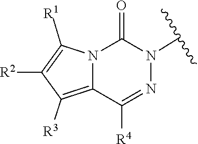

is

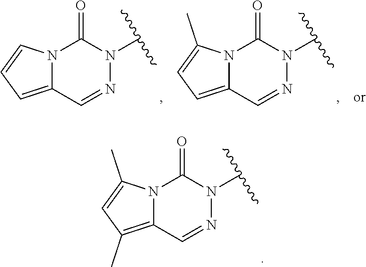

In a certain embodiment, R⁶ is H.

In a certain embodiment, R⁷ is —CH₃, —CH₂CH₃,

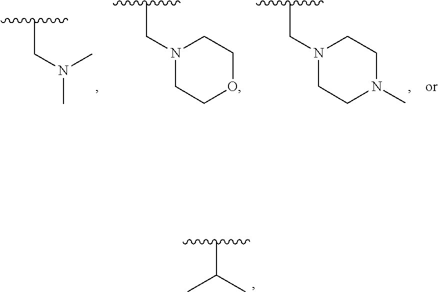

for example, —CH₃, —CH₂CH₃, or

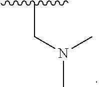 [SMILES: *CN(C)C]

In a certain embodiment, R⁸ is independently F, Cl, Br, —OH, —CH₃, —OCH₃, —OCF₃, —CF₃, or CN, for example, F, Cl, Br, —OH, —CH₃, —OCH₃, or —OCF₃.

In a certain embodiment,

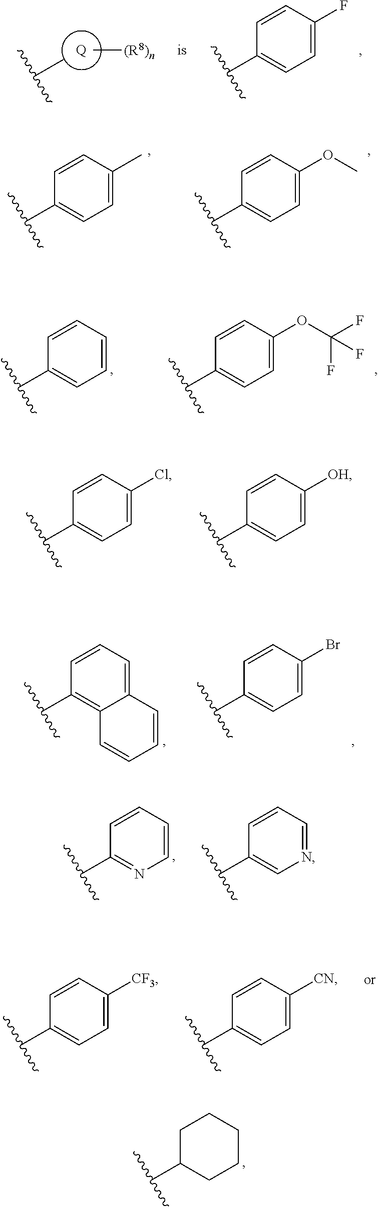

for example,

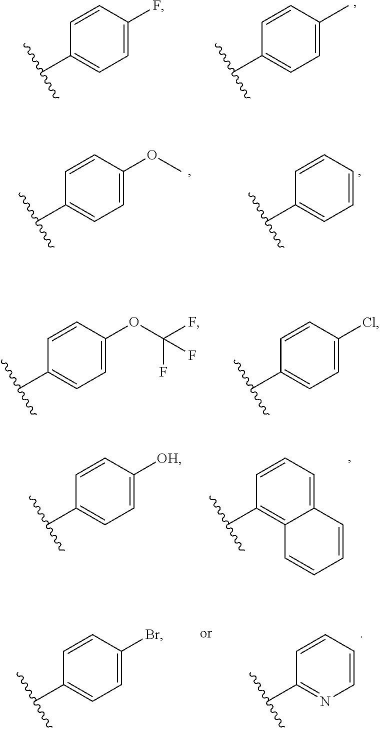

In a certain embodiment,

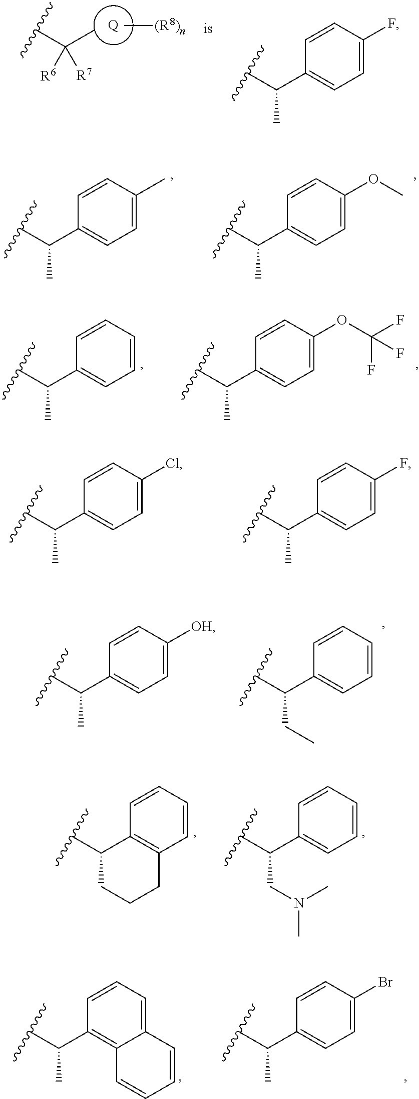

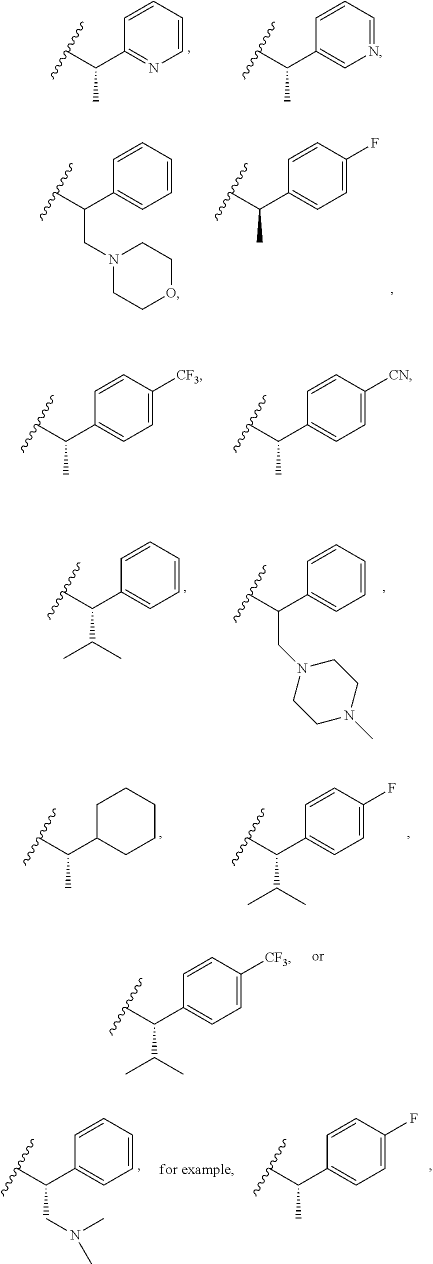

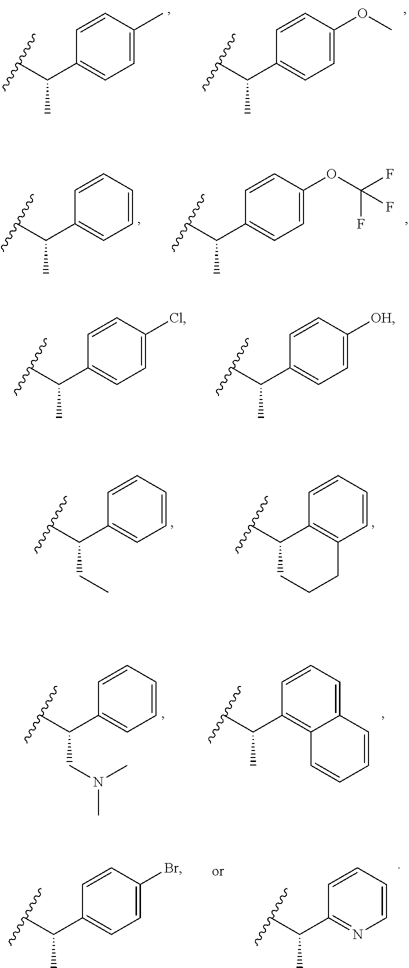

In a certain embodiment, the pyrrolotriazinone compound of formula I is selected from any one of the following compounds:

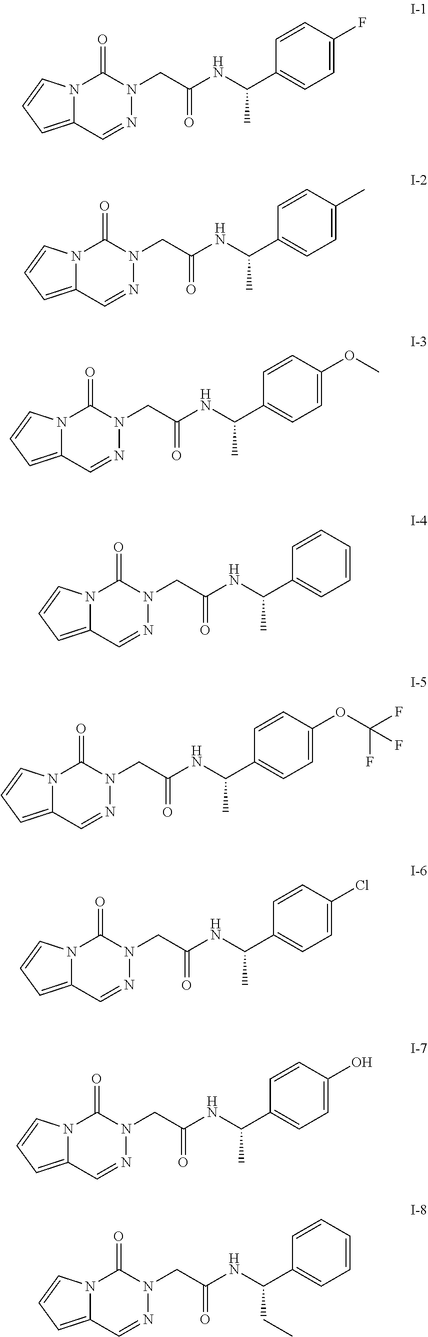

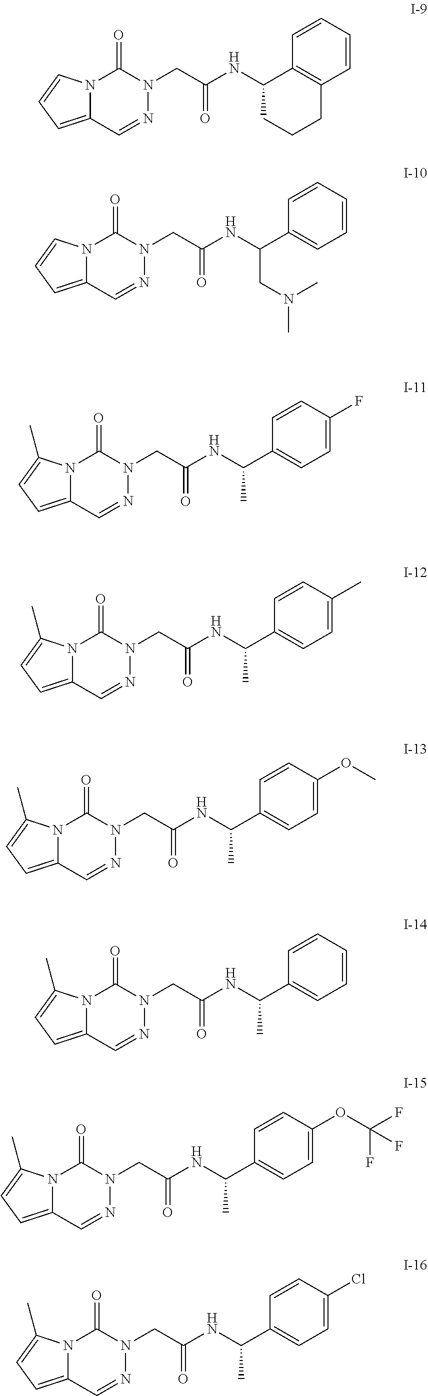

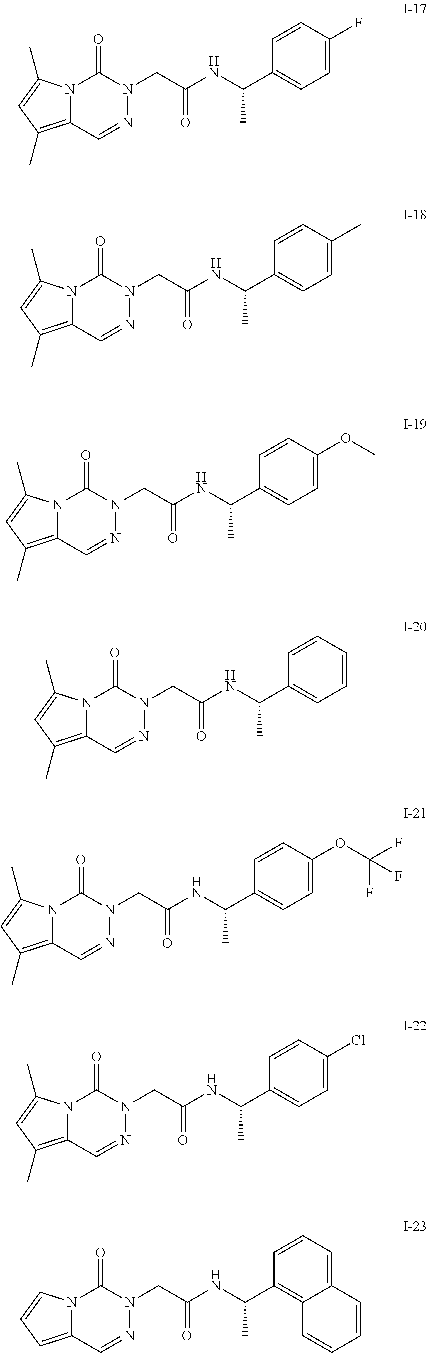

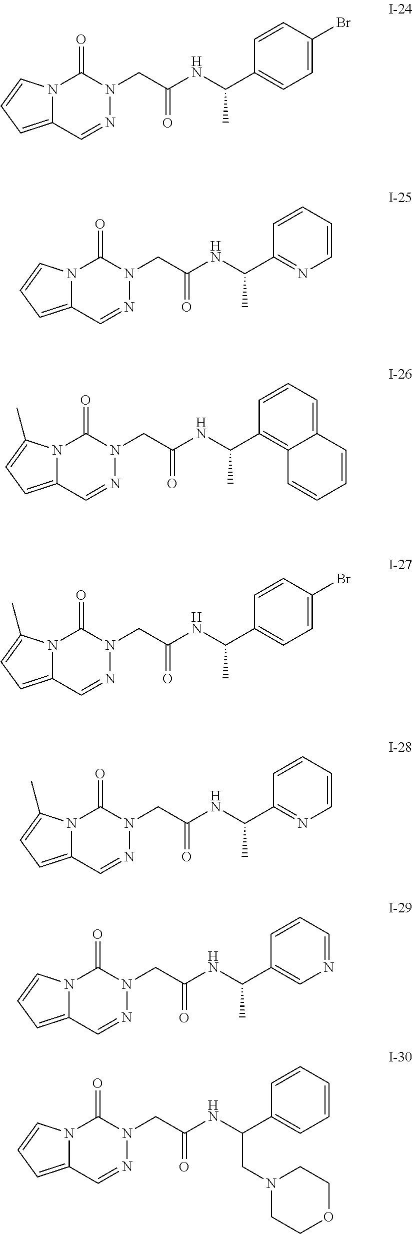

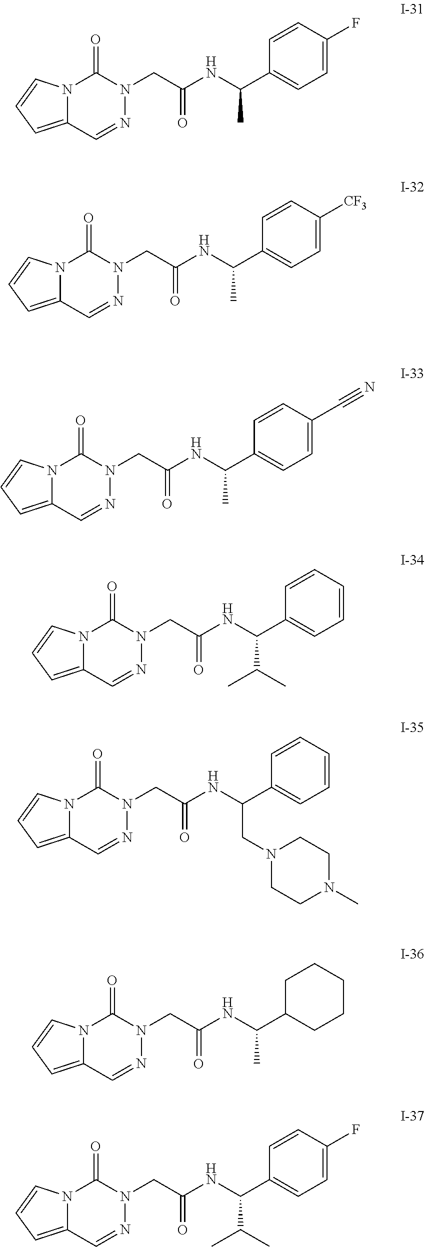

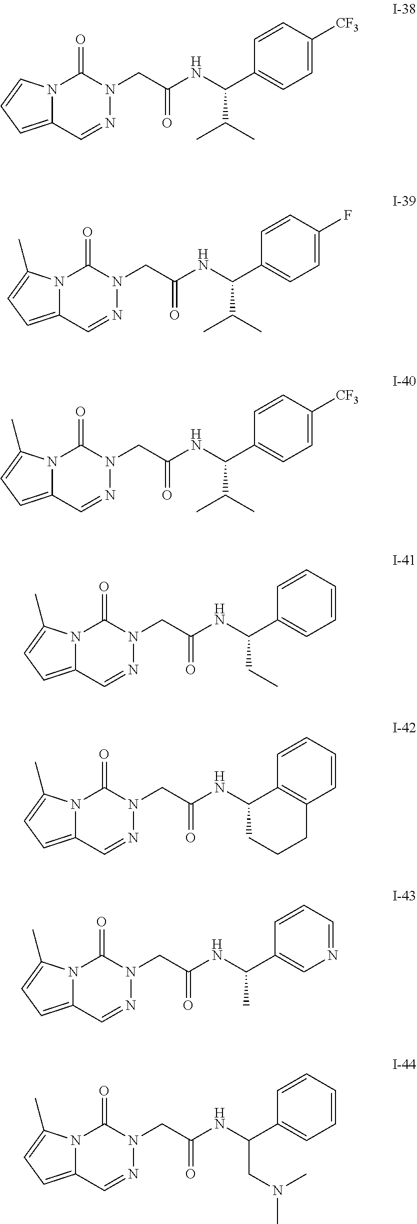

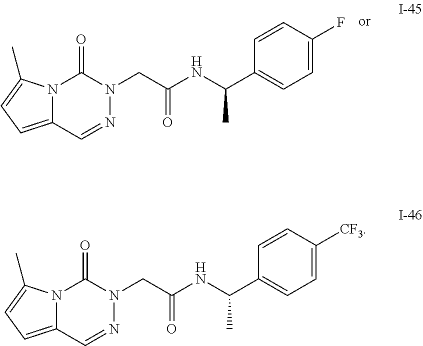 [SMILES: Cc1ccc2cnn(CC(=O)N[C@H](C)c3ccc(F)cc3)c(=O)n12 ; "or" ; Cc1ccc2cnn(CC(=O)N[C@@H](C)c3ccc(C(F)(F)F)cc3)c(=O)n12]

The present disclosure further provides a pharmaceutical composition, and the pharmaceutical composition comprises the pyrrolotriazinone compound of formula I or the pharmaceutically acceptable salt thereof, and a pharmaceutical excipient.

The present disclosure further provides a preparation method for the pyrrolotriazinone compound of formula I and the pharmaceutically acceptable salt thereof, and the preparation method comprises a following step: carrying out a condensation reaction between compound II and compound III;

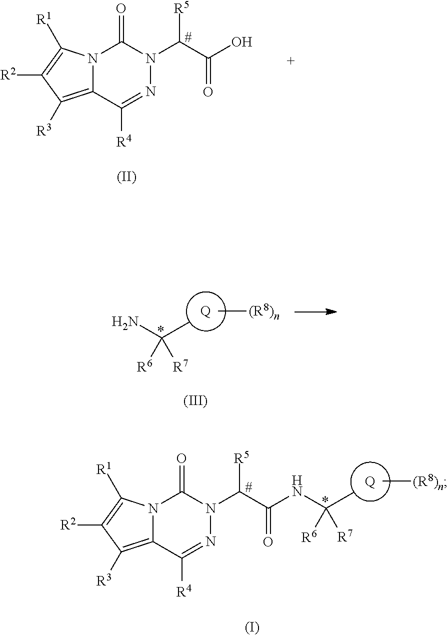

wherein *, #, R¹, R², R³, R⁴, R⁵, R⁶, R⁷, R⁸, Q, and n are defined as described in any one of the previous embodiments.

The present disclosure further provides a use of the pyrrolotriazinone compound of formula I and the pharmaceutically acceptable salt thereof or the pharmaceutical composition in the manufacture of a medicament for treating and/or preventing a GPR139 receptor-associated disease; the GPR139 receptor-associated disease may be schizophrenia, bipolar disorder, depression, cognitive disorder, autism spectrum disorder, sleep disorder, attention deficit hyperactivity disorder, post-traumatic stress disorder, substance abuse, drug addiction, eating disorder, obsessive-compulsive disorder, anxiety disorder, pain, or fibromyalgia.

The present disclosure further provides a use of the pyrrolotriazinone compound of formula I and the pharmaceutically acceptable salt thereof or the pharmaceutical composition in the manufacture of a medicament for treating and/or preventing schizophrenia, bipolar disorder, depression, cognitive disorder, autism spectrum disorder, sleep disorder, attention deficit hyperactivity disorder, post-traumatic stress disorder, substance abuse, drug addiction, eating disorder, obsessive-compulsive disorder, anxiety disorder, pain, or fibromyalgia.

Unless otherwise specified, the terms used in the present disclosure have the following meanings:

The term “halogen” refers to fluorine, chlorine, bromine, or iodine.

The term “alkyl” refers to a straight or branched chain alkyl group with a specified number of carbon atoms (e.g., C₁₋₆). Alkyl groups include, but are not limited to, methyl, ethyl, n-propyl, isopropyl, n-butyl, tert-butyl, isobutyl, sec-butyl, n-pentyl, n-hexyl, etc.

The term “cycloalkyl” refers to a cyclic group, composed solely of carbon atoms, with a specified number of carbon atoms (e.g., C₃₋₆). Cycloalkyl groups include, but are not limited to, cyclopropyl, cyclobutyl, cyclopentyl, cyclohexyl, etc.

The term “heterocycloalkyl” refers to a cyclic group with a specified number of ring atoms (e.g., 5- to 6-membered), a specified number of heteroatoms (e.g., 1, 2, or 3), and a specified type of heteroatoms (one or more kinds of N, O, and S). Heterocycloalkyl groups include, but are not limited to, azetidinyl, tetrahydropyrrolyl, tetrahydrofuryl, morpholinyl, piperidinyl, etc.

The term “aromatic ring” refers to a cyclic group composed solely of carbon atoms, with a specified number of carbon atoms (e.g., C₆₋₁₀). It can be monocyclic or polycyclic, with at least one ring being aromatic (conforming to Huckel's rule). Aromatic rings include but are not limited to benzene rings, naphthalene rings, etc.

The term “heteroaromatic ring” refers to a cyclic group with a specified number of ring atoms (e.g., 5- to 10-membered), a specified number of heteroatoms (e.g., 1, 2, or 3), and a specified type of heteroatoms (one or more kinds of N, O, and S). It can be monocyclic or polycyclic, with at least one ring being aromatic (conforming to Huckel's rule). Heteroaromatic rings are connected to other moieties in the molecule either through an aromatic or a non-aromatic ring. Heteroaromatic rings include, but are not limited to, furan rings, pyrrole rings, thiophene rings, pyrazole rings, imidazole rings, oxazole rings, thiazole rings, pyridine rings, pyrimidine rings, indole rings, etc.

The “-” at the end of a group indicates that the group is connected at that point to other moieties in the molecule. For example, —OCH₃ refers to methoxy.

When any variable (e.g., group R^{a-1}) appears multiple times in the definition of a compound, their definitions are independent of each other and do not affect each other. For example, 5- to 6-membered heterocycloalkyl substituted by 3 R^{a-1} means that the 5- to 6-membered heterocycloalkyl is substituted by 3 R^{a-1}, and the definitions of these 3 R^{a-1} are independent and do not affect each other.

The term “pharmaceutically acceptable salt” refers to salts derived from the reaction of a compound with a pharmaceutically acceptable acid or base, which are relatively non-toxic, safe, and suitable for patient use. When a compound contains relatively acidic functional groups, the corresponding base addition salts can be obtained by contacting the free form of the compound with a sufficient amount of pharmaceutically acceptable base in an appropriate inert solvent. Pharmaceutically acceptable base addition salts include, but are not limited to, sodium salts, potassium salts, calcium salts, aluminum salts, magnesium salts, bismuth salts, ammonium salts, etc. When a compound contains relatively basic functional groups, acid addition salts can be obtained by contacting the free form of the compound with a sufficient amount of pharmaceutically acceptable acid in an appropriate inert solvent. Pharmaceutically acceptable acid addition salts include, but are not limited to, hydrochloride salts, sulfate salts, methanesulfonate salts, etc. For details, refer to Handbook of Pharmaceutical Salts: Properties, Selection, and Use (P. Heinrich Stahl, 2002).

The term “therapeutically effective amount” refers to an amount of a compound administered to a patient that is sufficient to effectively treat a disease. The therapeutically effective amount will vary depending on the compound, type of disease, severity of the disease, patient's age, etc., but can be adjusted by those skilled in the art as required.

The term “pharmaceutical excipient” refers to the formulating agents and additives used in the manufacture of drugs and the compounding of prescriptions. They are substances contained in a pharmaceutical formulation, apart from the active ingredients. For details, refer to the Pharmacopoeia of the People's Republic of China (2020 Edition) or the Handbook of Pharmaceutical Excipients (Raymond C Rowe, 2009).

The term “treat/treatment” refers to any of the following situations: (1) alleviating one or more biological manifestations of a disease; (2) interfering with one or more points in the biological cascade that initiates the disease; (3) slowing the progression of one or more biological manifestations of the disease.

The term “prevent/prevention” refers to reducing the risk of developing a disease.

The term “patient” refers to any animal who has already received or is about to receive treatment, preferably a mammal, most preferably a human. Mammals include, but are not limited to, cattle, horses, sheep, pigs, cats, dogs, mice, rats, rabbits, guinea pigs, monkeys, and humans.

On the basis of not violating the common sense in the field, the preferred conditions above can be arbitrarily combined to obtain the preferred examples of the present disclosure.

The reagents and raw materials used in the present disclosure are commercially available.

The positive and progressive effect of the present disclosure is that the pyrrolotriazinone compound of the present disclosure has a novel structure and has strong agonistic activity on GPR139 receptors.

### BRIEF DESCRIPTION OF THE DRAWINGS

FIG. 1 is a graph of results in bioassay example 3.

### DETAILED DESCRIPTION OF THE PREFERRED EMBODIMENT

The present disclosure is further described below by the way of examples, but the present disclosure is not thereby limited to the scope of the described examples. In the following examples, experimental methods without specified conditions are carried out according to conventional methods and conditions, or are selected according to the product instructions.

### Example 1: Preparation of (S)—N-(1-(4-fluorophenyl)ethyl-2-(4-oxopyrrolo[1,2-d][1,2,4]triazin-3(4H)yl)acetamide (compound I-1)

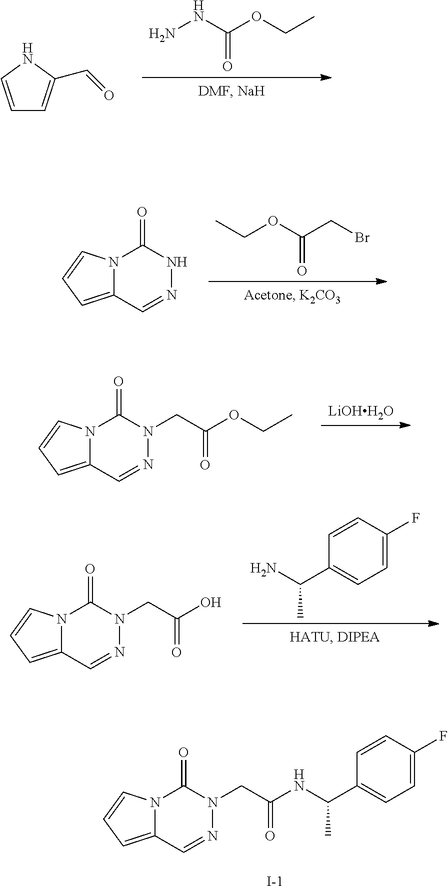

### Step 1: Preparation of pyrrolo[1,2-d][1,2,4]triazin-4(3H)-one

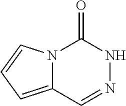

Pyrrole-2-carboxaldehyde (1.00 g, 10.52 mmol) and ethyl carbazate (1.20 g, 11.57 mmol) were dissolved in DMF (10 mL) and stirred at 90° C. for 24 hours. After cooling to room temperature, NaH (60% in mineral oil, 0.235 g, 5.26 mmol) was added thereto at 0° C., and the reaction mixture was stirred at 90° C. for 12 hours. The reaction mixture was diluted with water and extracted three times with ethyl acetate. The combined organic phases were washed with brine, dried over anhydrous Na₂SO₄, filtered, and concentrated under vacuum. The remaining solid was purified by silica gel column chromatography (0 to 20% EA/PE) to obtain the title compound (0.90 g, yield: 63%) as a yellow solid. ¹H NMR (800 MHZ, DMSO-d₆) § 12.24 (s, 1H), 8.24 (s, 1H), 7.77-7.75 (m, 1H), 6.84-6.82 (m, 2H). ¹³C NMR (201 MHz, DMSO-d₆) δ 144.72, 132.44, 125.60, 116.23, 115.43, 109.10. HRMS (ESI) calculated for C₆H₆N₃O₃⁺ [M+H]⁺: 136.0505; found: 136.0508.

### Step 2: Preparation of ethyl 2-(4-oxopyrrolo[1,2-d][1,2,4]triazin-3(4H)yl)acetate

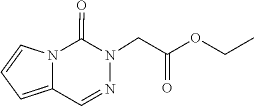 [SMILES: CCOC(=O)Cn1ncc2cccn2c1=O]

Potassium carbonate (1.17 g, 8.44 mmol) was dissolved in acetone solution (8 mL), and then pyrrolo[1,2-d][1,2,4]triazin-4(3H)-one (0.38 g, 2.81 mmol) was added thereto. Ethyl bromoacetate (0.939 g, 5.62 mmol) was added thereto under stirring, and the reaction mixture was stirred at 55° C. for 3 hours. The reaction mixture was diluted with water and extracted with ethyl acetate. The combined organic phases were washed with brine, dried over anhydrous Na₂SO₄, filtered, and concentrated under vacuum. The remaining solid was purified by silica gel column chromatography (0 to 5% EA/PE) to obtain the title compound (0.49 g, yield: 78%) as a light yellow oil. ¹H NMR (600 MHz, DMSO-d₆) δ 8.32 (s, 1H), 7.81 (d, J=2.5 Hz, 1H), 6.90 (dd, J=3.7, 1.4 Hz, 1H), 6.88-6.85 (m, 1H), 4.85 (s, 2H), 4.17 (q, J=7.0 Hz, 2H), 1.21 (t, J=7.2 Hz, 3H). HRMS (ESI) calculated for C₁₀H₁₂N₃O₃⁺ [M+H]⁺: 222.0873; found: 222.0875.

### Step 3: Preparation of 2-(4-oxopyrrolo[1,2-d][1,2,4]triazin-3(4H)yl)acetic Acid

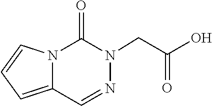 [SMILES: O=C(O)Cn1ncc2cccn2c1=O]

Ethyl 2-(4-oxopyrrolo[1,2-d][1,2,4]triazin-3(4H)yl)acetate (0.40 g, 1.81 mmol) was dissolved in a solution of THF:H₂O (1:1, 8 mL), then lithium hydroxide (0.227 g, 5.42 mmol) was added thereto at 0° C., and the reaction mixture was stirred at room temperature for 2 hours. Then, the pH of the reaction mixture was adjusted to about 2 using 1 M hydrochloric acid. The reaction mixture was evaporated to dryness under reduced pressure to remove the solvent to obtain the corresponding solid title compound (0.35 g, crude product), which was used without further purification. HRMS (ESI) calculated for C₈H₈N₃O₃⁺ [M+H]⁺: 194.0560; found: 194.0561.

### Step 4: Preparation of (S)—N-(1-(4-fluorophenyl)ethyl-2-(4-oxopyrrolo[1,2-d][1,2,4]triazin-3(4H)yl)acetamide (compound I-1)

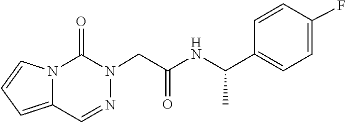 [SMILES: C[C@H](NC(=O)Cn1ncc2cccn2c1=O)c1ccc(F)cc1]

2-(4-Oxopyrrolo[1,2-d][1,2,4]triazin-3(4H)yl)acetic acid (0.100 g, 0.51 mmol) was dissolved in a solution of DMF (6 mL), then HATU (0.393 g, 1.03 mmol) was added thereto, and the reaction mixture was stirred at room temperature for 30 minutes. DIPEA (0.201 g, 1.55 mmol) and (S)-1-(4-fluorophenyl)ethylamine (0.108 g, 0.77 mmol) were sequentially added thereto and stirred at room temperature overnight. The reaction mixture was diluted with water and extracted twice with ethyl acetate. The combined organic phases were washed with brine, dried over anhydrous Na₂SO₄, filtered, and concentrated under vacuum. The remaining solid was purified by silica gel column chromatography (0 to 50% EA/PE) to obtain compound (I-1) (0.100 g, yield: 61%) as a white solid. ¹H NMR (800 MHZ, DMSO-d₆) δ 8.62 (d, J=7.9 Hz, 1H), 8.27 (s, 1H), 7.78 (d, J=2.2 Hz, 1H), 7.37-7.34 (m, 2H), 7.17-7.13 (m, 2H), 6.86 (dd, J=3.6, 1.4 Hz, 1H), 6.85-6.83 (m, 1H), 4.96-4.92 (m, 1H), 4.68 and 4.66 (ABq, J=16.7 Hz, 2H), 1.36 (d, J=7.0 Hz, 3H). ¹³C NMR (201 MHz, DMSO-d₆) δ 165.81, 161.02 (d, J=242.5 Hz), 144.59, 140.46 (d, J=3.3 Hz), 132.32, 127.88 (2C, d, J=8.1 Hz), 125.61, 117.02, 115.88, 114.91 (2C, d, J=20.9 Hz), 109.60, 52.91, 47.49, 22.36. HRMS (ESI) calculated for C₁₆H₁₆FN₄O₂⁺ [M+H]⁺: 315.1252; found: 315.1251.

### Example 2: Preparation of (S)-2-(4-oxopyrrolo[1,2-d][1,2,4]triazin-3(4H)yl)-N-(1-(p-tolyl)ethyl)acetamide (compound I-2)

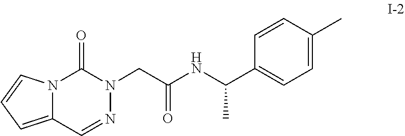 [SMILES: Cc1ccc([C@H](C)NC(=O)Cn2ncc3cccn3c2=O)cc1]

2-(4-Oxopyrrolo[1,2-d][1,2,4]triazin-3(4H)yl)acetic acid (0.15 g, 0.77 mmol) was dissolved in a solution of DMF (6 mL), then HATU (0.59 g, 1.55 mmol) was added thereto, and the reaction mixture was stirred at room temperature for 30 minutes. DIPEA (0.301 g, 2.33 mmol) and (S)-1-(4-methylphenyl)ethylamine (0.126 g, 0.93 mmol) were sequentially added thereto and stirred at room temperature overnight. The reaction mixture was diluted with water and extracted twice with ethyl acetate. The combined organic phases were washed with brine, dried over anhydrous Na₂SO₄, filtered, and concentrated under vacuum. The remaining solid was purified by silica gel column chromatography (0 to 50% EA/PE) to obtain compound (I-2) (0.092 g, yield: 38%) as a white solid. ¹H NMR (800 MHZ, DMSO-d₆) δ 8.56 (d, J=8.0 Hz, 1H), 8.27 (s, 1H), 7.78 (d, J=2.9 Hz, 1H), 7.20 (d, J=8.0 Hz, 2H), 7.13 (d, J=7.9 Hz, 2H), 6.86 (dd, J=3.7, 1.4 Hz, 1H), 6.84 (dd, J=3.7, 2.8 Hz, 1H), 4.91-4.88 (m, 1H), 4.67 and 4.65 (ABq, J=16.4 Hz, 2H), 2.27 (s, 3H), 1.35 (d, J=7.0 Hz, 3H). ¹³C NMR (201 MHZ, DMSO-d₆) δ 165.66, 144.59, 141.23, 135.70, 132.28, 128.76 (2C), 125.88 (2C), 125.61, 117.00, 115.86, 109.56, 52.88, 47.78, 22.36, 20.60. HRMS (ESI) calculated for C₁₇H₁₉N₄O₂⁺ [M+H]⁺: 311.1503; found: 311.1504.

### Example 3: Preparation of (S)—N-(1-(4-methoxyphenyl)ethyl)-2-(4-oxopyrrolo[1,2-d][1,2,4]triazin-3(4H)yl)acetamide (compound I-3)

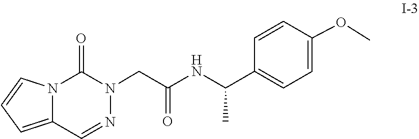 [SMILES: COc1ccc([C@H](C)NC(=O)Cn2ncc3cccn3c2=O)cc1]

The(S)-1-(4-methylphenyl)ethylamine in Example 2 was replaced with (S)-1-(4-methoxyphenyl)ethylamine, and the rest of the required raw materials, reagents, and preparation methods were the same as those in Example 2, yielding compound (I-3) (0.060 g, yield: 23%) as a white solid. ¹H NMR (800 MHZ, DMSO-d₆) δ 8.53 (d, J=8.0 Hz, 1H), 8.27 (s, 1H), 7.78 (d, J=2.2 Hz, 1H), 7.26-7.21 (m, 2H), 6.91-6.87 (m, 2H), 6.86 (dd, J=3.7, 1.4 Hz, 1H), 6.85-6.83 (m, 1H), 4.91-4.87 (m, 1H), 4.67 and 4.64 (ABq, J=16.3 Hz, 2H), 3.73 (s, 3H), 1.35 (d, J=6.9 Hz, 3H). ¹³C NMR (201 MHz, DMSO-d₆) δ 165.61, 158.07, 144.59, 136.17, 132.28, 127.12 (2C), 125.61, 117.01 (2C), 115.87, 113.61, 109.57, 55.05, 52.88, 47.44, 22.35. HRMS (ESI) calculated for C₁₇H₁₉N₄O₃⁺ [M+H]⁺: 327.1452; found: 327.1452.

### Example 4: Preparation of (S)-2-(4-oxopyrrolo[1,2-d][1,2,4]triazin-3(4H)yl)-N-(1-phenylethyl)acetamide (compound I-4)

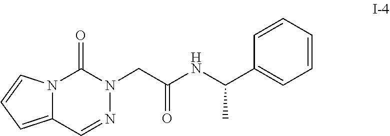 [SMILES: C[C@H](NC(=O)Cn1ncc2cccn2c1=O)c1ccccc1]

The(S)-1-(4-methylphenyl)ethylamine in Example 2 was replaced with (S)-1-phenylethylamine, and the rest of the required raw materials, reagents, and preparation methods were the same as those in Example 2, yielding compound (I-4) (0.070 g, yield: 30%) as a white solid. ¹H NMR (800 MHZ, DMSO-d₆) δ 8.62 (d, J=8.0 Hz, 1H), 8.27 (s, 1H), 7.78 (d, J=3.5 Hz, 1H), 7.34-7.30 (m, 4H), 7.25-7.21 (m, 1H), 6.86 (dd, J=3.6, 1.4 Hz, 1H), 6.84 (dd, J=3.7, 2.8 Hz, 1H), 4.96-4.92 (m, 1H), 4.69 and 4.67 (ABq, J=16.3 Hz, 2H), 1.37 (d, J=6.9 Hz, 3H). ¹³C NMR (201 MHz, DMSO-d₆) δ 165.76, 144.59, 144.25, 132.30, 128.24 (2C), 126.67, 125.94 (2C), 125.61, 117.02, 115.87, 109.58, 52.90, 48.06, 22.38. HRMS (ESI) calculated for C₁₆H₁₇N₄O₂⁺ [M+H]⁺: 297.1346; found: 297.1347.

### Example 5: Preparation of (S)-2-(4-oxopyrrolo[1,2-d][1,2,4]triazin-3(4H)yl)-N-(1-(4-(trifluoromethoxy)phenyl)ethyl)acetamide (compound I-5)

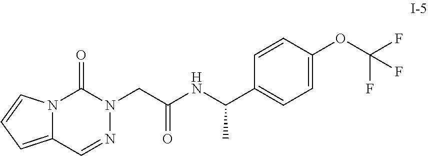 [SMILES: C[C@H](NC(=O)Cn1ncc2cccn2c1=O)c1ccc(OC(F)(F)F)cc1]

The(S)-1-(4-methylphenyl)ethylamine in Example 2 was replaced with (S)-1-4-(trifluoromethoxy)phenylethylamine, and the rest of the required raw materials, reagents, and preparation methods were the same as those in Example 2, yielding compound (I-5) (0.120 g, yield: 40%) as a white solid. ¹H NMR (800 MHZ, DMSO-d₆) δ 8.67 (d, J=7.8 Hz, 1H), 8.28 (s, 1H), 7.78 (d, J=2.3 Hz, 1H), 7.45 (d, J=8.7 Hz, 2H), 7.32 (d, J=8.3 Hz, 2H), 6.86 (dd, J=3.7, 1.4 Hz, 1H), 6.84 (t, J=3.3 Hz, 1H), 5.00-4.94 (m, 1H), 4.69 (s, 2H), 1.38 (d, J=7.0 Hz, 3H). ¹³C NMR (201 MHz, DMSO-d₆) δ 165.96, 147.04, 144.60, 143.81, 132.34, 127.82 (2C), 125.61, 120.89 (2C), 120.09 (q, J=256.3 Hz), 117.03, 115.88, 109.62, 52.92, 47.60, 22.23. HRMS (ESI) calculated for C₁₇H₁₆F₃N₄O₃⁺ [M+H]⁺: 381.1169; found: 381.1170.

### Example 6: Preparation of (S)—N-1-(4-chlorophenyl)ethyl-2-(4-oxopyrrolo[1,2-d][1,2,4]triazin-3(4H)yl)acetamide (compound I-6)

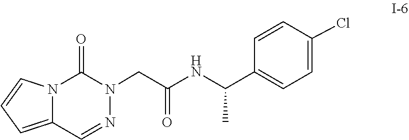 [SMILES: C[C@H](NC(=O)Cn1ncc2cccn2c1=O)c1ccc(Cl)cc1]

The(S)-1-(4-methylphenyl)ethylamine in Example 2 was replaced with (S)-1-(4-chlorophenyl)ethylamine hydrochloride, and the rest of the required raw materials, reagents, and preparation methods were the same as those in Example 2, yielding compound (I-6) (0.112 g, yield: 51%) as a white solid. ¹H NMR (800 MHZ, DMSO-d₆) δ 8.65 (d, J=7.8 Hz, 1H), 8.27 (s, 1H), 7.78 (d, J=1.8 Hz, 1H), 7.38 (d, J=8.5 Hz, 2H), 7.34 (d, J=8.5 Hz, 2H), 6.86 (d, J=2.2 Hz, 1H), 6.84 (t, J=3.3 Hz, 1H), 4.95-4.90 (m, 1H), 4.69 and 4.67 (ABq, J=16.6 Hz, 2H), 1.36 (d, J=7.0 Hz, 3H). ¹³C NMR (201 MHz, DMSO-d₆) δ 165.90, 144.58, 143.36, 132.32, 131.20, 128.17 (2C), 127.86 (2C), 125.60, 117.02, 115.88, 109.60, 52.91, 47.60, 22.19. HRMS (ESI) calculated for C₁₆H₁₆ClN₄O₂⁺ [M+H]⁺: 331.0956, found: 331.0955.

### Example 7: Preparation of (S)—N-(1-(4-hydroxyphenyl)ethyl)-2-(4-oxopyrrolo[1,2-d][1,2,4]triazin-3(4H)yl)acetamide (compound I-7)

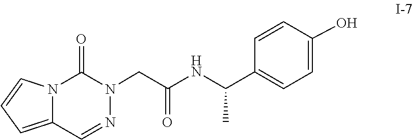 [SMILES: C[C@H](NC(=O)Cn1ncc2cccn2c1=O)c1ccc(O)cc1]

The(S)-1-(4-methylphenyl)ethylamine in Example 2 was replaced with (S)-4-(1-aminoethyl) phenol, and the rest of the required raw materials, reagents, and preparation methods were the same as those in Example 2.

Compound (I-7) (0.074 g, yield: 45%) as a white solid was obtained. ¹H NMR (800 MHz, DMSO-d₆) δ 9.26 (s, 1H), 8.48 (d, J=8.0 Hz, 1H), 8.27 (s, 1H), 7.78 (d, J=1.5 Hz, 1H), 7.13-7.10 (m, 2H), 6.86 (dd, J=3.7, 1.4 Hz, 1H), 6.84 (dd, J=3.7, 2.9 Hz, 1H), 6.72-6.69 (m, 2H), 4.88-4.83 (m, 1H), 4.65 and 4.63 (ABq, J=16.4 Hz, 2H), 1.33 (d, J=7.0 Hz, 3H). ¹³C NMR (201 MHz, DMSO-d₆) δ 165.55, 156.14, 144.61, 134.33, 132.29, 127.14 (2C), 125.63, 117.03, 115.89, 114.92 (2C), 109.58, 52.89, 47.46, 22.33. HRMS (ESI) calculated for C₁₆H₁₇N₄O₃⁺ [M+H]⁺: 313.1295, found: 313.1298.

### Example 8: Preparation of (S)-2-(4-oxopyrrolo[1,2-d][1,2,4]triazin-3(4H)yl)-N-(1-phenylpropyl)acetamide (compound I-8)

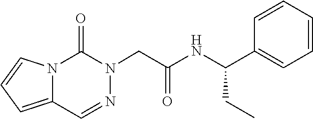 [SMILES: CC[C@H](NC(=O)Cn1ncc2cccn2c1=O)c1ccccc1]

The(S)-1-(4-methylphenyl)ethylamine in Example 2 was replaced with (S)-1-phenylpropylamine, and the rest of the required raw materials, reagents, and preparation methods were the same as those in Example 2, yielding compound (I-8) (0.095 g, yield: 59%) as a white solid. ¹H NMR (800 MHZ, DMSO-d₆) δ 8.55 (d, J=8.5 Hz, 1H), 8.27 (s, 1H), 7.78 (d, J=2.2 Hz, 1H), 7.34-7.29 (m, 4H), 7.25-7.22 (m, 1H), 6.86 (dd, J=3.6, 1.4 Hz, 1H), 6.84 (dd, J=3.7, 2.9 Hz, 1H), 4.74-4.69 (m, 2H), 4.67 (d, J=16.3 Hz, 1H), 1.74-1.66 (m, 2H), 0.86 (t, J=7.4 Hz, 3H). ¹³C NMR (201 MHz, DMSO-d₆) δ 166.11, 144.59, 143.32, 132.28, 128.21 (2C), 126.71, 126.42 (2C), 125.62, 117.02, 115.88, 109.58, 54.29, 52.91, 29.22, 10.98. HRMS (ESI) calculated for C₁₇H₁₉N₄O₂⁺ [M+H]⁺: 311.1503, found: 311.1501.

### Example 9: Preparation of (S)-2-(4-oxopyrrolo[1,2-d][1,2,4]triazin-3(4H)yl)-N-1,2,3,4-tetrahydronaphthalen-1-yl)acetamide (compound I-9)

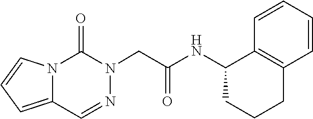 [SMILES: O=C(Cn1ncc2cccn2c1=O)N[C@H]1CCCc2ccccc21]

The(S)-1-(4-methylphenyl)ethylamine in Example 2 was replaced with (S)-1,2,3,4-tetrahydro-1-naphthylamine, and the rest of the required raw materials, reagents, and preparation methods were the same as those in Example 2, yielding compound (I-9) (0.100 g, yield: 59%) as a white solid. ¹H NMR (800 MHZ, DMSO-d₆) δ 8.55 (d, J=8.6 Hz, 1H), 8.30 (s, 1H), 7.81 (d, J=2.9 Hz, 1H), 7.22-7.18 (m, 1H), 7.20-7.13 (m, 2H), 7.11-7.07 (m, 1H), 6.87 (dd, J=3.6, 1.4 Hz, 1H), 6.85 (t, J=3.2 Hz, 1H), 5.02-4.97 (m, 1H), 4.70 and 4.62 (ABq, J=16.4 Hz, 2H), 2.78-2.68 (m, 2H), 1.93-1.84 (m, 2H), 1.77-1.65 (m, 2H). ¹³C NMR (201 MHz, DMSO-d₆) δ 166.15, 144.63, 137.18, 137.05, 132.37, 128.74, 128.12, 126.79, 125.86, 125.69, 117.04, 115.87, 109.57, 53.12, 46.74, 29.81, 28.73, 20.06. HRMS (ESI) calculated for C₁₈H₁₉N₄O₂⁺ [M+H]⁺: 322.1503, found: 322.1502.

### Example 10: Preparation of N-(2-(dimethylamino)-1-phenylethyl)-2-(4-oxopyrrolo[1,2-d][1,2,4]triazin-3(4H)yl)acetamide (compound I-10)

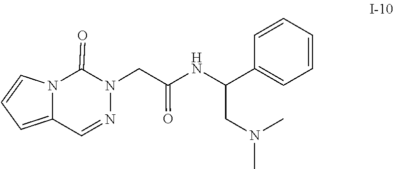 [SMILES: CN(C)CC(NC(=O)Cn1ncc2cccn2c1=O)c1ccccc1]

The(S)-4-(1-aminoethyl) phenol in Example 7 was replaced with N1,N1-dimethyl-2-phenylethane-1,2-diamine, and the rest of the required raw materials, reagents, and preparation methods were the same as those in Example 7, yielding compound (I-10) (0.050 g, yield: 28%) as a brown foamy solid. ¹H NMR (800 MHZ, DMSO-d₆) δ 8.80 (d, J=9.0 Hz, 1H), 8.30 (s, 1H), 7.79 (d, J=2.3 Hz, 1H), 7.46-7.39 (m, 4H), 7.34 (t, J=6.9 Hz, 1H), 6.88 (dd, J=3.7, 1.4 Hz, 1H), 6.86 (t, J=3.6 Hz, 1H), 5.41-5.27 (m, 1H), 4.82 and 4.76 (ABq, J=16.6 Hz, 2H), 3.48-3.32 (m, 2H), 2.82 (brs, 6H). ¹³C NMR (201 MHz, DMSO-d₆) δ 166.03, 144.58, 142.08, 132.28, 128.20 (2C), 126.87, 126.66 (2C), 125.58, 117.04, 115.91, 109.62, 64.44, 52.84, 50.95, 45.14 (2C). HRMS (ESI) calculated for C₁₈H₂₂N₅O₂⁺ [M+H]⁺: 340.1768, found: 340.1767.

### Example 11: Preparation of (S)—N-(1-(4-fluorophenyl)ethyl-2-(6-methyl-4-oxopyrrolo[1,2-d][1,2,4]triazin-3(4H)yl)acetamide (compound I-11)

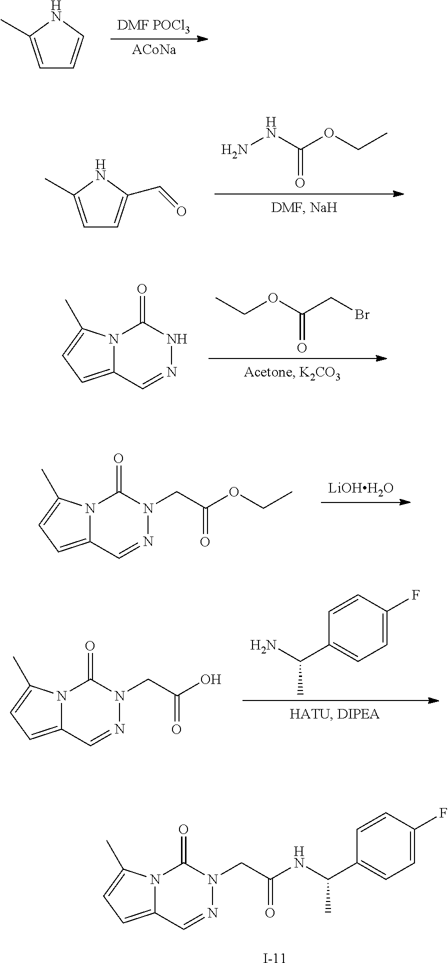

### Step 1: Preparation of 5-methylpyrrole-2-carbaldehyde

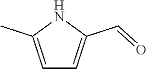

Dichloromethane (12 mL) was added to a three-necked round-bottomed flask, and then DMF (0.854 g, 11.7 mmol) was added thereto, and the system was replaced with nitrogen. POCl₃ (1.79 g, 11.7 mmol) was added dropwise thereto at 0° C. and stirred at room temperature for 15 minutes, then 2-methylpyrrole (1.0 g, 12.3 mmol) was added thereto at 0° C. and stirred at room temperature for 30 minutes. Anhydrous sodium acetate (4.6 g, 56.0 mmol) was dissolved in 13 mL of water, slowly added to a round-bottomed flask at room temperature, and stirred at 80° C. for 20 minutes. The reaction mixture was diluted with water and extracted twice with dichloromethane. The combined organic phases were washed with brine, dried over anhydrous Na₂SO₄, filtered, and concentrated under vacuum. The remaining solid was purified by silica gel column chromatography (0 to 20% EA/PE) to obtain the title compound (0.600 g, yield: 44%) as a light yellow solid. ¹H NMR (800 MHZ, DMSO-d₆) δ 11.86 (s, 1H), 9.30 (s, 1H), 6.89-6.86 (m, 1H), 6.03-5.99 (m, 1H), 2.24 (s, 3H). HRMS (ESI) calculated for C₆H₈NO⁺ [M+H]⁺: 110.0600, found: 110.0599.

### Step 2: Preparation of 6-methylpyrrolo[1,2-d][1,2,4]triazin-4-one

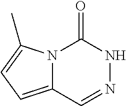

5-Methylpyrrole-2-carbaldehyde (2.00 g, 18.33 mmol) and ethyl carbazate (2.29 g, 21.99 mmol) were dissolved in DMF (20 mL) and stirred at 90° C. for 24 hours. After cooling to room temperature, NaH (60% in mineral oil, 0.733 g, 9.16 mmol) was added thereto at 0° C., and the reaction mixture was stirred at 90° C. for 12 hours. The reaction mixture was diluted with water and extracted three times with ethyl acetate. The combined organic phases were washed with brine, dried over anhydrous Na₂SO₄, filtered, and concentrated under vacuum. The remaining solid was purified by silica gel column chromatography (0 to 10% EA/PE) to obtain the title compound (1.70 g, yield: 62%) as a yellow solid. ¹H NMR (800 MHZ, DMSO-d₆) δ 11.82 (s, 1H), 8.04 (s, 1H), 6.64 (d, J=3.6 Hz, 1H), 6.47 (d, J=3.6 Hz, 1H), 2.69 (s, 3H). HRMS (ESI) calculated for C₇H₈N₃O⁺ [M+H]⁺: 150.0662, found: 150.0662.

### Step 3: Preparation of ethyl 2-(6-methyl-4-oxopyrrolo[1,2-d][1,2,4]triazin-3(4H)yl)acetate

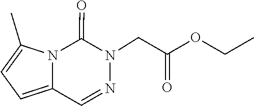 [SMILES: CCOC(=O)Cn1ncc2ccc(C)n2c1=O]

Potassium carbonate (4.73 g, 34.19 mmol) was dissolved in acetone solution (35 mL), and then 6-methylpyrrolo[1,2-d][1,2,4]triazin-4-one (1.70 g, 11.40 mmol) was added to the above solution. Ethyl bromoacetate (3.81 g, 5.62 mmol) was added thereto under stirring, and the reaction mixture was stirred at 55° C. overnight. The reaction mixture was diluted with water and extracted with ethyl acetate. The combined organic phases were washed with brine, dried over anhydrous Na₂SO₄, filtered, and concentrated under vacuum. The remaining solid was purified by silica gel column chromatography (0 to 5% EA/PE) to obtain the title compound (2.25 g, yield: 81%) as a light yellow solid. ¹H NMR (800 MHZ, DMSO-d₆) δ 8.13 (s, 1H), 6.71 (d, J=3.7 Hz, 1H), 6.53 (dd, J=3.7, 1.0 Hz, 1H), 4.74 (s, 2H), 4.17 and 4.15 (ABq, J=7.2 Hz, 2H), 2.68 (s, 3H), 1.21 (t, J=7.1 Hz, 3H). HRMS (ESI) calculated for C₁₁H₁₄N₃O₃⁺ [M+H]⁺: 236.1030, found: 236.1029.

### Step 4: Preparation of 2-(6-methyl-4-oxopyrrolo[1,2-d][1,2,4]triazin-3(4H)yl)acetic Acid

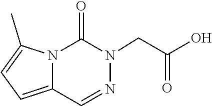

Ethyl 2-(6-methyl-4-oxopyrrolo[1,2-d][1,2,4]triazin-3(4H)yl)acetate (2.25 g, 9.56 mmol) was dissolved in a solution of THF:H₂O (1:1, 20 mL), then lithium hydroxide (1.20 g, 28.69 mmol) was added thereto at 0° C., and the reaction mixture was stirred at room temperature for 2 hours. The pH of the reaction mixture was adjusted to about 2 using 1 M hydrochloric acid. The reaction mixture was evaporated to dryness under reduced pressure to remove the solvent to obtain the corresponding solid title compound (1.7 g, crude product), which was used without further purification. HRMS (ESI) calculated for C₉H₁₀N₃O₃⁺ [M+H]⁺: 208.0717, found: 208.0717.

### Step 5: Preparation of (S)—N-(1-(4-fluorophenyl)ethyl-2-(6-methyl-4-oxopyrrolo[1,2-d][1,2,4]triazin-3(4H)yl)acetamide (compound I-11)

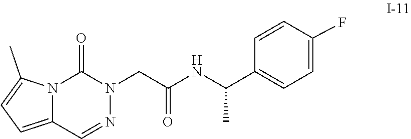 [SMILES: Cc1ccc2cnn(CC(=O)N[C@@H](C)c3ccc(F)cc3)c(=O)n12]

2-(6-Methyl-4-oxopyrrolo[1,2-d][1,2,4]triazin-3(4H)yl)acetic acid (0.120 g, 0.57 mmol) was dissolved in a solution of DMF (7 mL), then HATU (0.440 g, 1.14 mmol) was added thereto, and the reaction mixture was stirred at room temperature for 30 minutes. DIPEA (0.187 g, 1.71 mmol) and (S)-1-(4-fluorophenyl)ethylamine (0.089 g, 0.62 mmol) were sequentially added thereto and stirred at room temperature overnight. The reaction mixture was diluted with water and extracted twice with ethyl acetate. The combined organic phases were washed with brine, dried over anhydrous Na₂SO₄, filtered, and concentrated under vacuum. The remaining solid was purified by silica gel column chromatography (0 to 50% EA/PE) to obtain compound (I-11) (0.110 g, yield: 57%) as a white solid. ¹H NMR (800 MHZ, DMSO-d₆) δ 8.58 (d, J=7.9 Hz, 1H), 8.08 (s, 1H), 7.38-7.34 (m, 2H), 7.17-7.12 (m, 2H), 6.67 (d, J=3.7 Hz, 1H), 6.51 (dd, J=3.7, 1.0 Hz, 1H), 4.96-4.91 (m, 1H), 4.58 and 4.56 (ABq, J=16.5 Hz, 2H), 2.69 (s, 3H), 1.36 (d, J=7.0 Hz, 3H). ¹³C NMR (201 MHz, DMSO-d₆) δ 166.02, 161.03 (d, J=242.1 Hz), 146.22, 140.54 (d, J=2.9 Hz), 132.65, 130.75, 127.90 (2C, d, J=8.1 Hz), 125.73, 115.42, 114.91 (2C, d, J=21.3 Hz), 108.28, 52.70, 47.48, 22.43, 14.67. HRMS (ESI) calculated for C₁₇H₁₈FN₄O₂⁺ [M+H]⁺: 329.1408, found: 329.1409.

### Example 12: Preparation of (S)-2-(6-methyl-4-oxopyrrolo[1,2-d][1,2,4]triazin-3(4H)yl)-N-(1-(p-tolyl)ethyl)acetamide (compound I-12)

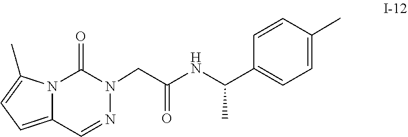 [SMILES: Cc1ccc([C@H](C)NC(=O)Cn2ncc3ccc(C)n3c2=O)cc1]

The(S)-1-(4-fluorophenyl)ethylamine in step 5 of Example 11 was replaced with (S)-1-(4-methylphenyl)ethylamine, and the rest of the required raw materials, reagents, and preparation methods were the same as those in step 5 of Example 11, yielding compound (I-12) (0.080 g, yield: 42%) as a white solid. ¹H NMR (800 MHZ, DMSO-d₆) δ 8.52 (d, J=7.9 Hz, 1H), 8.07 (s, 1H), 7.20 (d, J=8.0 Hz, 2H), 7.13 (d, J=7.7 Hz, 2H), 6.67 (d, J=3.6 Hz, 1H), 6.51 (dd, J=3.7, 1.0 Hz, 1H), 4.92-4.87 (m, 1H), 4.57 and 4.55 (ABq, J=16.4 Hz, 2H), 2.69 (s, 3H), 2.27 (s, 3H), 1.35 (d, J=7.0 Hz, 3H). ¹³C NMR (201 MHz, DMSO-d₆) δ 165.88, 146.23, 141.32, 135.70, 132.62, 130.74, 128.77 (2C), 125.90 (2C), 125.74, 115.41, 108.26, 52.67, 47.77, 22.43, 20.61, 14.68. HRMS (ESI) calculated for C₁₈H₂₁N₄O₂⁺ [M+H]⁺: 325.1659, found: 325.1659.

### Example 13: Preparation of (S)—N-(1-(4-methoxyphenyl)ethyl-2-(6-methyl-4-oxopyrrolo[1,2-d][1,2,4]triazin-3(4H)yl)acetamide (compound I-13)

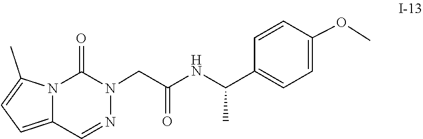 [SMILES: COc1ccc([C@H](C)NC(=O)Cn2ncc3ccc(C)n3c2=O)cc1]

The(S)-1-(4-fluorophenyl)ethylamine in step 5 of Example 11 was replaced with (S)-1-(4-methoxyphenyl)ethylamine, and the rest of the required raw materials, reagents, and preparation methods were the same as those in step 5 of Example 11, yielding compound (I-13) (0.118 g, yield: 59%) as a white solid. ¹H NMR (800 MHZ, DMSO-d₆) δ 8.49 (d, J=8.0 Hz, 1H), 8.07 (s, 1H), 7.25-7.22 (m, 2H), 6.90-6.87 (m, 2H), 6.67 (d, J=3.6 Hz, 1H), 6.51 (dd, J=3.7, 1.0 Hz, 1H), 4.92-4.87 (m, 1H), 4.57 and 4.54 (ABq, J=16.3 Hz, 2H), 3.73 (s, 3H), 2.69 (s, 3H), 1.35 (d, J=6.9 Hz, 3H). ¹³C NMR (201 MHz, DMSO-d₆) δ 165.81, 158.07, 146.23, 136.26, 132.62, 130.74, 127.14 (2C), 125.73, 115.41, 113.61 (2C), 108.25, 55.06, 52.65, 47.44, 22.43, 14.68. HRMS (ESI) calculated for C₁₈H₂₀NaN₄O₃⁺ [M+Na]⁺: 363.1428, found: 363.1428.

### Example 14: Preparation of (S)-2-(6-methyl-4-oxopyrrolo[1,2-d][1,2,4]triazin-3(4H)yl)-N-(1-phenylethyl)acetamide (compound I-14)

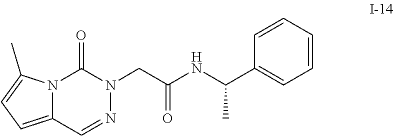 [SMILES: Cc1ccc2cnn(CC(=O)N[C@@H](C)c3ccccc3)c(=O)n12]

The(S)-1-(4-fluorophenyl)ethylamine in step 5 of Example 11 was replaced with (S)-1-phenylethylamine, and the rest of the required raw materials, reagents, and preparation methods were the same as those in step 5 of Example 11, yielding compound (I-14) (0.085 g, yield: 47%) as a white solid. ¹H NMR (800 MHZ, DMSO-d₆) δ 8.57 (d, J=7.9 Hz, 1H), 8.08 (s, 1H), 7.35-7.30 (m, 4H), 7.25-7.21 (m, 1H), 6.67 (d, J=3.6 Hz, 1H), 6.51 (dd, J=3.6, 1.0 Hz, 1H), 4.97-4.91 (m, 1H), 4.59 and 4.57 (ABq, J=16.3 Hz, 2H), 2.69 (s, 3H), 1.37 (d, J=7.0 Hz, 3H). ¹³C NMR (201 MHz, DMSO-d₆) δ 165.97, 146.23, 144.34, 132.63, 130.74, 128.25 (2C), 126.67, 125.96 (2C), 125.73, 115.42, 108.26, 52.68, 48.05, 22.45, 14.67. HRMS (ESI) calculated for C₁₇H₁₉N₄O₂⁺ [M+H]⁺: 311.1503, found: 311.1503.

### Example 15: Preparation of (S)-2-(6-methyl-4-oxopyrrolo[1,2-d][1,2,4]triazin-3(4H)yl)-N-(1-(4-(trifluoromethoxy)phenyl)ethyl)acetamide (compound I-15)

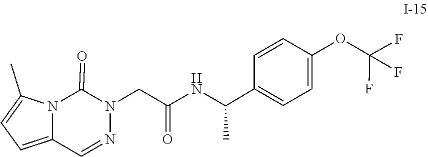 [SMILES: Cc1ccc2cnn(CC(=O)N[C@@H](C)c3ccc(OC(F)(F)F)cc3)c(=O)n12]

The(S)-1-(4-fluorophenyl)ethylamine in step 5 of Example 11 was replaced with (S)-1-4-(trifluoromethoxy)phenylethylamine, and the rest of the required raw materials, reagents, and preparation methods were the same as those in step 5 of Example 11, yielding compound (I-15) (0.125 g, yield: 54%) as a white solid. ¹H NMR (800 MHZ, DMSO-d₆) δ 8.64 (d, J=7.7 Hz, 1H), 8.08 (s, 1H), 7.46-7.43 (m, 2H), 7.32 (d, J=7.9 Hz, 2H), 6.67 (d, J=3.6 Hz, 1H), 6.51 (dd, J=3.6, 1.0 Hz, 1H), 4.99-4.94 (m, 1H), 4.59 (s, 2H), 2.69 (s, 3H), 1.38 (d, J=7.0 Hz, 3H). ¹³C NMR (201 MHz, DMSO-d₆) δ 166.17, 147.04, 146.23, 143.90, 132.68, 130.76, 127.84 (2C), 125.73, 120.90 (2C), 120.11 (q, J=255.7 Hz), 115.43, 108.31, 52.72, 47.60, 22.28, 14.66. HRMS (ESI) calculated for C₁₈H₁₇NaF₃N₄O₃⁺ [M+Na]⁺: 417.1145, found: 417.1145.

### Example 16: Preparation of (S)—N-1-(4-chlorophenyl)ethyl-2-(6-methyl-4-oxopyrrolo[1,2-d][1,2,4]triazin-3(4H)yl)acetamide (compound I-16)

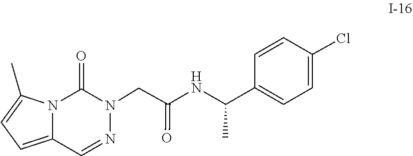 [SMILES: Cc1ccc2cnn(CC(=O)N[C@@H](C)c3ccc(Cl)cc3)c(=O)n12]

The(S)-1-(4-fluorophenyl)ethylamine in step 5 of Example 11 was replaced with (S)-1-(4-chlorophenyl)ethylamine hydrochloride, and the rest of the required raw materials, reagents, and preparation methods were the same as those in step 5 of Example 11, yielding compound (I-16) (0.088 g, yield: 44%) as a white solid. ¹H NMR (800 MHZ, DMSO-d₆) δ 8.61 (d, J=7.8 Hz, 1H), 8.08 (s, 1H), 7.40-7.37 (m, 2H), 7.36-7.33 (m, 2H), 6.67 (d, J=3.6 Hz, 1H), 6.51 (dd, J=3.6, 1.0 Hz, 1H), 4.95-4.90 (m, 1H), 4.59 and 4.57 (ABq, J=16.3 Hz, 2H), 2.69 (s, 3H), 1.36 (d, J=7.0 Hz, 3H). ¹³C NMR (201 MHz, DMSO-d₆) δ 166.11, 146.22, 143.44, 132.66, 131.20, 130.75, 128.17 (2C), 127.88 (2C), 125.72, 115.42, 108.29, 52.70, 47.59, 22.26, 14.67. HRMS (ESI) calculated for C₁₇H₁₈C₁₄N₄O₂⁺ [M+H]⁺: 345.1113, found: 345.1118.

### Example 17: Preparation of (S)-2-(6,8-dimethyl-4-oxopyrrolo[1,2-d][1,2,4]triazin-3(4H)yl)-N-(1-(4-fluorophenyl)ethyl)acetamide (compound I-17)

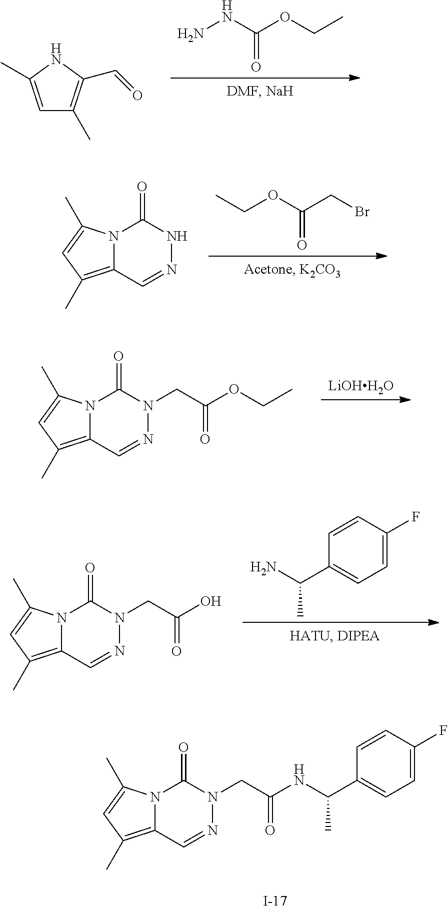

### Step 1: Preparation of 6,8-dimethylpyrrolo[1,2-d][1,2,4]triazin-4-one

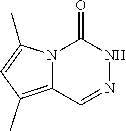

3,5-Dimethyl-2-pyrrolecarboxaldehyde (1.00 g, 8.12 mmol) and ethyl carbazate (1.01 g, 9.74 mmol) were dissolved in DMF (10 mL) and stirred at 90° C. for 24 hours. After cooling to room temperature, NaH (60% in mineral oil, 0.325 g, 8.12 mmol) was added thereto at 0° C., and the reaction mixture was stirred at 90° C. for 12 hours. The reaction mixture was diluted with water and extracted three times with ethyl acetate. The combined organic phases were washed with brine, dried over anhydrous Na₂SO₄, filtered, and concentrated under vacuum. The remaining solid was purified by silica gel column chromatography (0 to 10% EA/PE) to obtain the title compound (0.85 g, yield: 64%) as a light yellow solid. ¹H NMR (800 MHZ, DMSO-d₆) δ 11.64 (s, 1H), 8.07 (s, 1H), 6.32 (s, 1H), 2.65 (s, 3H), 2.20 (s, 3H). ¹³C NMR (201 MHz, DMSO-d₆) δ 146.49, 131.66, 129.34, 122.65, 117.86, 116.86, 14.33, 10.01. HRMS (ESI) calculated for C₈H₁₀N₃O⁺ [M+H]⁺: 164.0818; found: 164.0817.

### Step 2: Preparation of ethyl 2-(6,8-dimethyl-4-oxopyrrolo[1,2-d][1,2,4]triazin-3(4H)yl)acetate

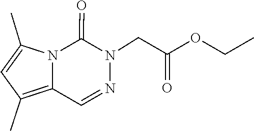 [SMILES: CCOC(=O)Cn1ncc2c(C)cc(C)n2c1=O]

Potassium carbonate (4.32 g, 31.25 mmol) was dissolved in acetone solution (35 mL), and then 6,8-dimethylpyrrolo[1,2-d][1,2,4]triazin-4-one (1.70 g, 10.42 mmol) was added to the above solution. Ethyl bromoacetate (3.48 g, 20.84 mmol) was added thereto under stirring, and the reaction mixture was stirred at 55° C. overnight. The reaction mixture was diluted with water and extracted with ethyl acetate. The combined organic phases were washed with brine, dried over anhydrous Na₂SO₄, filtered, and concentrated under vacuum. The remaining solid was purified by silica gel column chromatography (0 to 5% EA/PE) to obtain the title compound (2.20 g, yield: 84%) as a yellow solid. ¹H NMR (800 MHZ, DMSO-d₆) δ 8.17 (s, 1H), 6.39 (s, 1H), 4.71 (s, 2H), 4.17 and 4.15 (ABq, J=7.2 Hz, 2H), 2.64 (s, 3H), 2.22 (s, 3H), 1.21 (t, J=7.1 Hz, 3H). HRMS (ESI) calculated for C₁₂H₁₆N₃O₃⁺ [M+H]⁺: 250.1186; found: 250.1186.

### Step 3: Preparation of 2-(6,8-dimethyl-4-oxopyrrolo[1,2-d][1,2,4]triazin-3(4H)yl)acetic Acid

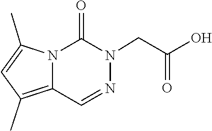 [SMILES: Cc1cc(C)n2c(=O)n(CC(=O)O)ncc12]

Ethyl 2-(6,8-dimethyl-4-oxopyrrolo[1,2-d][1,2,4]triazin-3(4H)yl)acetate (2.10 g, 8.42 mmol) was dissolved in a solution of THF:H₂O (1:1, 10 mL), then lithium hydroxide (1.06 g, 25.27 mmol) was added thereto at 0° C., and the reaction mixture was stirred at room temperature for 2 hours. The pH of the reaction mixture was adjusted to about 2 using 1 M hydrochloric acid. The reaction mixture was evaporated to dryness under reduced pressure to remove the solvent to obtain the corresponding solid title compound (1.8 g, crude product), which was used without further purification. HRMS (ESI) calculated for C₁₀H₁₂N₃O₃⁺ [M+H]⁺: 222.0873; found: 222.0874.

### Step 4: Preparation of (S)-2-(6,8-dimethyl-4-oxopyrrolo[1,2-d][1,2,4]triazin-3(4H)yl)-N-(1-(4-fluorophenyl)ethyl)acetamide (compound I-17)

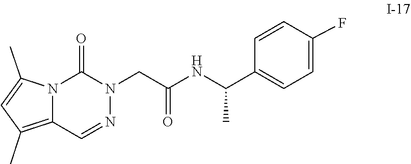 [SMILES: Cc1cc(C)n2c(=O)n(CC(=O)N[C@@H](C)c3ccc(F)cc3)ncc12]

2-(6,8-Dimethyl-4-oxopyrrolo[1,2-d][1,2,4]triazin-3(4H)yl)acetic acid (0.200 g, 0.90 mmol) was dissolved in a solution of DMF (7 mL), then HATU (0.688 g, 1.80 mmol) was added thereto, and the reaction mixture was stirred at room temperature for 30 minutes. DIPEA (0.292 g, 2.25 mmol) and (S)-1-(4-fluorophenyl)ethylamine (0.138 g, 0.99 mmol) were sequentially added thereto and stirred at room temperature overnight. The reaction mixture was diluted with water and extracted twice with ethyl acetate. The combined organic phases were washed with brine, dried over anhydrous Na₂SO₄, filtered, and concentrated under vacuum. The remaining solid was purified by silica gel column chromatography (0 to 50% EA/PE) to obtain compound I-17 (0.160 g, yield: 51%) as a white solid. ¹H NMR (800 MHZ, DMSO-d₆) δ 8.55 (d, J=7.9 Hz, 1H), 8.11 (s, 1H), 7.39-7.32 (m, 2H), 7.14 (t, J=8.9 Hz, 2H), 6.36 (s, 1H), 4.97-4.91 (m, 1H), 4.55 and 4.53 (ABq, J=16.4 Hz, 2H), 2.64 (s, 3H), 2.21 (s, 3H), 1.36 (d, J=7.0 Hz, 3H). ¹³C NMR (201 MHz, DMSO-d₆) δ 166.13, 161.02 (2C, d, J=242.5 Hz), 146.22, 140.56, 131.60, 130.04, 127.90 (2C, d, J=8.1 Hz), 122.83, 118.39, 117.29, 114.91 (d, J=21.3 Hz), 52.55, 47.44, 22.43, 14.49, 9.97. HRMS (ESI) calculated for C₁₈H₂₀FN₄O₂⁺ [M+H]⁺: 343.1565; found: 343.1567.

### Example 18: Preparation of (S)-2-(6,8-dimethyl-4-oxopyrrolo[1,2-d][1,2,4]triazin-3(4H)yl)-N-(1-(p-tolyl)ethyl)acetamide (compound I-18)

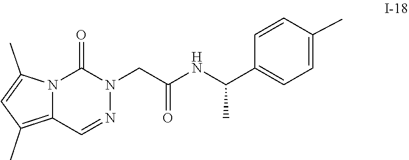 [SMILES: Cc1ccc([C@H](C)NC(=O)Cn2ncc3c(C)cc(C)n3c2=O)cc1]

The(S)-1-(4-fluorophenyl)ethylamine in step 4 of Example 17 was replaced with (S)-1-(4-methylphenyl)ethylamine, and the rest of the required raw materials, reagents, and preparation methods were the same as those in step 4 of Example 17, yielding compound (I-18) (0.180 g, yield: 58%) as a white solid. 1H NMR (800 MHZ, DMSO-d₆) δ 8.49 (d, J=8.0 Hz, 1H), 8.10 (s, 1H), 7.20 (d, J=8.0 Hz, 2H), 7.12 (d, J=7.9 Hz, 2H), 6.36 (s, 1H), 4.93-4.86 (m, 1H), 4.54 and 4.52 (ABq, J=16.2 Hz, 2H), 2.64 (s, 3H), 2.27 (s, 3H), 2.21 (s, 3H), 1.35 (d, J=7.0 Hz, 3H). ¹³C NMR (201 MHz, DMSO-d₆) δ 165.97, 146.21, 141.34, 135.68, 131.56, 130.02, 128.76 (2C), 125.90 (2C), 122.83, 118.35, 117.28, 52.52, 47.73, 22.43, 20.61, 14.49, 9.96. HRMS (ESI) calculated for C₁₉H₂₃N₄O₂⁺ [M+H]⁺: 339.1816; found: 339.1818.

### Example 19: Preparation of (S)-2-(6,8-dimethyl-4-oxopyrrolo[1,2-d][1,2,4]triazin-3(4H)yl)-N-(1-(4-methoxyphenyl)ethyl)acetamide (compound I-19)

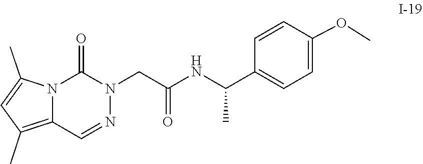 [SMILES: COc1ccc([C@H](C)NC(=O)Cn2ncc3c(C)cc(C)n3c2=O)cc1]

The(S)-1-(4-fluorophenyl)ethylamine in step 4 of Example 17 was replaced with (S)-1-(4-methoxyphenyl)ethylamine, and the rest of the required raw materials, reagents, and preparation methods were the same as those in step 4 of Example 17, yielding compound (I-19) (0.150 g, yield: 46%) as a light yellow solid. ¹H NMR (800 MHZ, DMSO-d₆) δ 8.47 (d, J=8.0 Hz, 1H), 8.10 (s, 1H), 7.27-7.19 (m, 2H), 6.90-6.86 (m, 2H), 6.35 (s, 1H), 4.92-4.87 (m, 1H), 4.54 and 4.51 (ABq, J=14.3 Hz, 2H), 3.73 (s, 3H), 2.64 (d, J=0.9 Hz, 3H), 2.21 (s, 3H), 1.35 (d, J=6.9 Hz, 3H). ¹³C NMR (201 MHz, DMSO-d₆) δ 165.90, 158.05, 146.20, 136.27, 131.53, 130.01, 127.13 (2C), 122.82, 118.33, 117.25, 113.59 (2C), 55.05, 52.50, 47.39, 22.40, 14.46, 9.94. HRMS (ESI) calculated for C₁₉H₂₃N₄O₃⁺ [M+H]⁺: 355.1765; found: 355.1764.

### Example 20: Preparation of (S)-2-(6,8-dimethyl-4-oxopyrrolo[1,2-d][1,2,4]triazin-3(4H)yl)-N-(1-phenylethyl)acetamide (compound I-20)

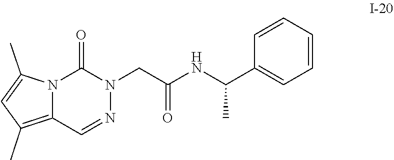 [SMILES: Cc1cc(C)n2c(=O)n(CC(=O)N[C@@H](C)c3ccccc3)ncc12]

The(S)-1-(4-fluorophenyl)ethylamine in step 4 of Example 17 was replaced with (S)-1-phenylethylamine, and the rest of the required raw materials, reagents, and preparation methods were the same as those in step 4 of Example 17, yielding compound (I-20) (0.130 g, yield: 44%) as a white solid. ¹H NMR (800 MHZ, DMSO-d₆) δ 8.55 (d, J=8.0 Hz, 1H), 8.11 (s, 1H), 7.32 (d, J=4.4 Hz, 4H), 7.26-7.20 (m, 1H), 6.35 (s, 1H), 4.97-4.92 (m, 1H), 4.56 and 4.54 (ABq, J=16.2 Hz, 2H), 2.64 (s, 3H), 2.21 (s, 3H), 1.37 (d, J=7.0 Hz, 3H). ¹³C NMR (201 MHz, DMSO-d₆) δ 166.06, 146.20, 144.34, 131.55, 130.01, 128.22 (2C), 126.64, 125.95 (2C), 122.82, 118.34, 117.25, 52.52, 48.00, 22.42, 14.46, 9.94. HRMS (ESI) calculated for C₁₈H₂₁N₄O₂⁺ [M+H]⁺: 325.1659; found: 325.1659.

### Example 21: Preparation of (S)-2-(6,8-dimethyl-4-oxopyrrolo[1,2-d][1,2,4]triazin-3(4H)yl)-N-(1-(4-(trifluoromethoxy)phenyl)ethyl)acetamide (compound I-21)

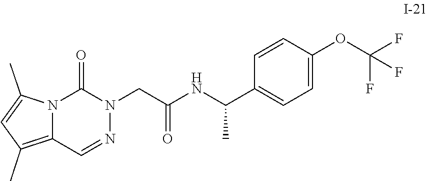 [SMILES: Cc1cc(C)n2c(=O)n(CC(=O)N[C@@H](C)c3ccc(OC(F)(F)F)cc3)ncc12]

The(S)-1-(4-fluorophenyl)ethylamine in step 4 of Example 17 was replaced with (S)-1-4-(trifluoromethoxy)phenylethylamine, and the rest of the required raw materials, reagents, and preparation methods were the same as those in step 4 of Example 17, yielding compound (I-21) (0.240 g, yield: 65%) as a white solid. ¹H NMR (800 MHZ, DMSO-d₆) δ 8.61 (d, J=7.7 Hz, 1H), 8.11 (s, 1H), 7.47-7.43 (m, 2H), 7.32 (d, J=8.1 Hz, 2H), 6.36 (s, 1H), 5.00-4.94 (m, 1H), 4.56 (s, 2H), 2.64 (s, 3H), 2.21 (s, 3H), 1.38 (d, J=7.0 Hz, 3H). ¹³C NMR (201 MHz, DMSO-d₆) δ 166.26, 147.02, 146.20, 143.89, 131.59, 130.04, 127.83 (2C), 122.82, 120.87 (2C), 120.10 (q, J=256.7 Hz), 118.38, 117.27, 52.55, 47.54, 22.25, 14.44, 9.93. HRMS (ESI) calculated for C₁₉H₂₀F₃N₄O₃⁺ [M+H]⁺: 409.1482; found: 409.1483.

### Example 22: Preparation of (S)—N-1-(4-chlorophenyl)ethyl-2-(6,8-dimethyl-4-oxopyrrolo[1,2-d][1,2,4]triazin-3(4H)yl)acetamide (compound I-22)

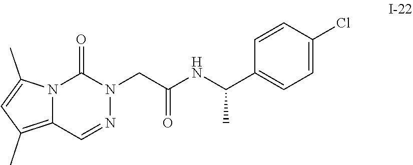 [SMILES: Cc1cc(C)n2c(=O)n(CC(=O)N[C@@H](C)c3ccc(Cl)cc3)ncc12]

The(S)-1-(4-fluorophenyl)ethylamine in step 4 of Example 17 was replaced with (S)-1-(4-chlorophenyl)ethylamine hydrochloride, and the rest of the required raw materials, reagents, and preparation methods were the same as those in step 4 of Example 17, yielding compound (I-22) (0.140 g, yield: 43%) as a white solid. ¹H NMR (800 MHZ, DMSO-d₆) δ 8.58 (d, J=7.8 Hz, 1H), 8.11 (s, 1H), 7.40-7.36 (m, 2H), 7.36-7.33 (m, 2H), 6.36 (s, 1H), 4.95-4.89 (m, 1H), 4.55 and 4.53 (ABq, J=16.3 Hz, 2H), 2.64 (s, 3H), 2.21 (s, 3H), 1.36 (d, J=7.0 Hz, 3H). ¹³C NMR (201 MHz, DMSO-d₆) δ 166.22, 146.21, 143.47, 131.61, 131.18, 130.05, 128.17 (2C), 127.89 (2C), 122.83, 118.40, 117.29, 52.55, 47.56, 22.26, 14.48, 9.97. HRMS (ESI) calculated for C₁₈H₂₀ClN₄O₂⁺ [M+H]⁺: 359.1269; found: 359.1268.

### Example 23: Preparation of (S)—N-(1-(naphthalen-1-ethyl)-2-(4-oxopyrrolo[1,2-d][1,2,4]triazin-3(4H)yl)acetamide (compound I-23)

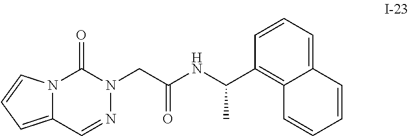 [SMILES: C[C@H](NC(=O)Cn1ncc2cccn2c1=O)c1cccc2ccccc12]

The(S)-1-(4-methylphenyl)ethylamine in Example 2 was replaced with (S)-1-(1-naphthyl)ethylamine, and the rest of the required raw materials, reagents, and preparation methods were the same as those in Example 2, yielding compound (I-23) (0.130 g, yield: 72%) as a white solid. ¹H NMR (800 MHZ, DMSO-d₆) δ 8.74 (d, J=7.9 Hz, 1H), 8.28 (s, 1H), 7.90-7.87 (m, 3H), 7.83-7.78 (m, 2H), 7.53-7.49 (m, 2H), 7.49-7.47 (m, 1H), 6.86 (dd, J=3.6, 1.4 Hz, 1H), 6.84 (t, J=3.3 Hz, 1H), 5.14-5.09 (m, 1H), 4.73 and 4.71 (ABq, J=16.3 Hz, 2H), 1.47 (d, J=7.0 Hz, 3H). ¹³C NMR (201 MHz, DMSO-d₆) δ 165.94, 144.62, 141.74, 132.85, 132.33, 132.06, 127.89, 127.65, 127.45, 126.15, 125.65, 125.63, 124.89, 124.00, 117.03, 115.89, 109.60, 53.01, 48.24, 22.18. HRMS (ESI) calculated for C₂₀H₁₉N₄O₂⁺ [M+H]⁺: 347.1503, found: 347.1507.

### Example 24: Preparation of (S)—N-(1-(4-bromophenyl)ethyl-2-(4-oxopyrrolo[1,2-d][1,2,4]triazin-3(4H)yl)acetamide (compound I-24)

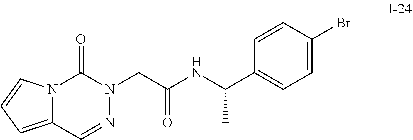 [SMILES: C[C@H](NC(=O)Cn1ncc2cccn2c1=O)c1ccc(Br)cc1]

The(S)-1-(4-methylphenyl)ethylamine in Example 2 was replaced with (S)-1-(4-bromophenyl)ethylamine, and the rest of the required raw materials, reagents, and preparation methods were the same as those in Example 2, yielding compound (I-24) (0.130 g, yield: 66%) as a white solid. ¹H NMR (800 MHZ, DMSO-d₆) δ 8.65 (d, J=7.7 Hz, 1H), 8.27 (s, 1H), 7.78 (d, J=2.1 Hz, 1H), 7.53-7.50 (m, 2H), 7.30-7.26 (m, 2H), 6.86 (dd, J=3.6, 1.4 Hz, 1H), 6.84 (t, J=3.3 Hz, 1H), 4.93-4.88 (m, 1H), 4.68 and 4.65 (ABq, J=16.8 Hz, 2H), 1.35 (d, J=7.0 Hz, 3H). ¹³C NMR (201 MHz, DMSO-d₆) δ 165.93, 144.60, 143.83, 132.35, 131.12 (2C), 128.27 (2C), 125.61, 119.69, 117.05, 115.91, 109.64, 52.92, 47.70, 22.18. HRMS (ESI) calculated for C₁₆H₁₆BrN₄O₂⁺ [M+H]⁺: 375.0451, found: 375.0450.

### Example 25: Preparation of (S)-2-(4-oxopyrrolo[1,2-d][1,2,4]triazin-3(4H)yl)-N-(1-pyridin-2-yl)ethyl)acetamide (compound I-25)

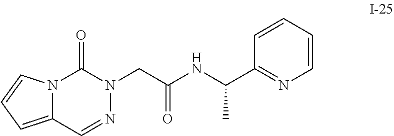 [SMILES: C[C@H](NC(=O)Cn1ncc2cccn2c1=O)c1ccccn1]

The(S)-1-(4-methylphenyl)ethylamine in Example 2 was replaced with (S)-1-(pyridin-2-yl)ethylamine, and the rest of the required raw materials, reagents, and preparation methods were the same as those in Example 2, yielding compound (I-25) (0.110 g, yield: 71%) as a white solid. ¹H NMR (800 MHZ, DMSO-d₆) δ 8.69 (d, J=7.9 Hz, 1H), 8.52 (dd, J=4.7, 0.9 Hz, 1H), 8.28 (s, 1H), 7.79 (dd, J=1.5, 0.8 Hz, 1H), 7.78-7.75 (m, 1H), 7.37 (d, J=7.9 Hz, 1H), 7.28-7.25 (m, 1H), 6.86 (dd, J=3.7, 1.4 Hz, 1H), 6.85-6.83 (m, 1H), 5.01-4.96 (m, 1H), 4.73 and 4.70 (ABq, J=16.4 Hz, 2H), 1.40 (d, J=7.0 Hz, 3H). ¹³C NMR (201 MHz, DMSO-d₆) δ 166.11, 162.20, 148.79, 144.61, 136.81, 132.34, 125.62, 122.14, 120.30, 117.03, 115.90, 109.62, 52.95, 49.91, 21.08. HRMS (ESI) calculated for C₁₅H₁₆NsO₂⁺ [M+H]⁺: 298.1299, found: 298.1299.

### Example 26: Preparation of (S)-2-(6-methyl-4-oxopyrrolo[1,2-d][1,2,4]triazin-3(4H)yl)-N-(1-naphthalen-1-yl)ethyl)acetamide (compound I-26)

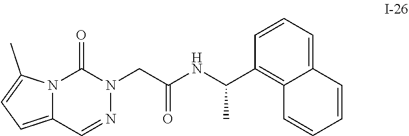 [SMILES: Cc1ccc2cnn(CC(=O)N[C@@H](C)c3cccc4ccccc34)c(=O)n12]

The(S)-1-(4-fluorophenyl)ethylamine in step 5 of Example 11 was replaced with (S)-1-(1-naphthyl)ethylamine, and the rest of the required raw materials, reagents, and preparation methods were the same as those in step 5 of Example 11, yielding compound (I-26) (0.130 g, yield: 74%) as a white solid. ¹H NMR (800 MHZ, DMSO-d₆) δ 8.71 (d, J=7.8 Hz, 1H), 8.09 (s, 1H), 7.90-7.86 (m, 3H), 7.82 (d, J=2.0 Hz, 1H), 7.52-7.49 (m, 2H), 7.49-7.46 (m, 1H), 6.67 (d, J=3.6 Hz, 1H), 6.51 (dd, J=3.7, 1.0 Hz, 1H), 5.13-5.08 (m, 1H), 4.64 and 4.61 (ABq, J=16.4 Hz, 2H), 2.70 (s, 3H), 1.47 (d, J=7.0 Hz, 3H). ¹³C NMR (201 MHz, DMSO-d₆) δ 166.17, 146.25, 141.82, 132.86, 132.65, 132.05, 130.73, 127.87, 127.63, 127.46, 126.13, 125.75, 125.64, 124.92, 123.98, 115.42, 108.27, 52.85, 48.22, 22.22, 14.68. HRMS (ESI) calculated for C₂₁H₂₁N₄O₂⁺ [M+H]⁺: 361.1659, found: 361.1656.

### Example 27: Preparation of (S)—N-1-(4-bromophenyl)ethyl-2-(6-methyl-4-oxopyrrolo[1,2-d][1,2,4]triazin-3(4H)yl)acetamide (compound I-27)

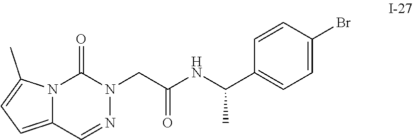 [SMILES: Cc1ccc2cnn(CC(=O)N[C@@H](C)c3ccc(Br)cc3)c(=O)n12]

The(S)-1-(4-fluorophenyl)ethylamine in step 3 of Example 11 was replaced with (S)-1-(4-bromophenyl)ethylamine, and the rest of the required raw materials, reagents, and preparation methods were the same as those in step 5 of Example 11, yielding compound (I-27) (0.140 g, yield: 74%) as a white solid. ¹H NMR (800 MHZ, DMSO-d₆) δ 8.62 (d, J=7.8 Hz, 1H), 8.08 (s, 1H), 7.54-7.49 (m, 2H), 7.30-7.27 (m, 2H), 6.67 (d, J=3.6 Hz, 1H), 6.51 (dd, J=3.7, 1.0 Hz, 1H), 4.93-4.88 (m, 1H), 4.59 and 4.56 (ABq, J=16.3 Hz, 2H), 2.69 (s, 3H), 1.35 (d, J=7.0 Hz, 3H). ¹³C NMR (201 MHz, DMSO-d₆) δ 166.12, 146.21, 143.89, 132.66, 131.09 (2C), 130.75, 128.27 (2C), 125.72, 119.67, 115.42, 108.29, 52.69, 47.66, 22.21, 14.67. HRMS (ESI) calculated for C₁₇H₁₈BᵣN₄O₂⁺ [M+H]⁺: 388.0608, found: 388.0609.

### Example 28: Preparation of (S)-2-(6-methyl-4-oxopyrrolo[1,2-d][1,2,4]triazin-3(4H)yl)-N-(1-(pyridin-2-yl)ethyl)acetamide (compound I-28)

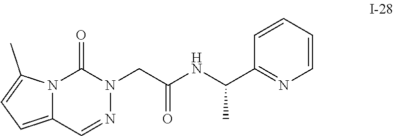 [SMILES: Cc1ccc2cnn(CC(=O)N[C@@H](C)c3ccccn3)c(=O)n12]

The 11 step 3 in step 3 of Example 11 was replaced with (S)-1-(pyridin-2-yl)ethylamine, and the rest of the required raw materials, reagents, and preparation methods were the same as those in step 3 of Example 11, yielding compound (I-28) (0.080 g, yield: 53%) as a white solid. ¹H NMR (800 MHZ, DMSO-d₆) δ 8.65 (d, J=7.8 Hz, 1H), 8.52 (d, J=5.7 Hz, 1H), 8.08 (s, 1H), 7.77 (td, J=7.6, 1.8 Hz, 1H), 7.38 (d, J=7.8 Hz, 1H), 7.28-7.24 (m, 1H), 6.67 (d, J=3.6 Hz, 1H), 6.51 (dd, J=3.7, 1.0 Hz, 1H), 5.01-4.96 (m, 1H), 4.63 and 4.60 (ABq, J=16.3 Hz, 2H), 2.69 (s, 3H), 1.40 (d, J=7.0 Hz, 3H). ¹³C NMR (201 MHz, DMSO-d₆) δ 166.31, 162.26, 148.77, 146.23, 136.79, 132.65, 130.74, 125.72, 122.13, 120.31, 115.43, 108.29, 52.73, 49.87, 21.11, 14.67. HRMS (ESI) calculated for C₁₆H₁₈N₅O₂⁺ [M+H]⁺: 312.1455, found: 312.1454.

### Example 29: Preparation of (S)-2-(4-oxopyrrolo[1,2-d][1,2,4]triazin-3(4H)yl)-N-(1-(pyridin-3-yl)ethyl)acetamide (compound I-29)

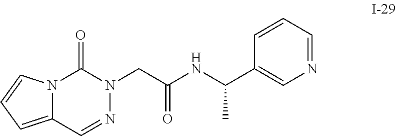 [SMILES: C[C@H](NC(=O)Cn1ncc2cccn2c1=O)c1cccnc1]

The(S)-4-(1-aminoethyl) phenol in Example 7 was replaced with (S)-1-(pyridin-3-yl)ethylamine, and the rest of the required raw materials, reagents, and preparation methods were the same as those in Example 7, yielding compound (I-29) (0.064 g, yield: 41%) as a white solid. ¹H NMR (800 MHZ, DMSO-d₆) δ 8.71 (d, J=7.7 Hz, 1H), 8.54 (d, J=2.4 Hz, 1H), 8.45 (dd, J=4.8, 1.7 Hz, 1H), 8.28 (s, 1H), 7.79 (d, J=2.2 Hz, 1H), 7.73-7.71 (m, 1H), 7.37-7.35 (m, 1H), 6.86 (dd, J=3.6, 1.4 Hz, 1H), 6.84 (t, J=3.3 Hz, 1H), 5.00-4.96 (m, 1H), 4.70 and 4.68 (ABq, J=16.6 Hz, 2H), 1.41 (d, J=7.0 Hz, 3H). ¹³C NMR (201 MHz, DMSO-d₆) δ 166.08, 148.00, 147.74, 144.61, 139.51, 133.58, 132.37, 125.62, 123.41, 117.05, 115.92, 109.65, 52.97, 46.26, 21.98. HRMS (ESI) calculated for C₁₅H₁₆NsO₂⁺ [M+H]⁺: 298.1299, found: 298.1295.

### Example 30: Preparation of N-(2-morpholino-1-phenylethyl)-2-(4-oxopyrrolo[1,2-d][1,2,4]triazin-3(4H)yl)acetamide (compound I-30)

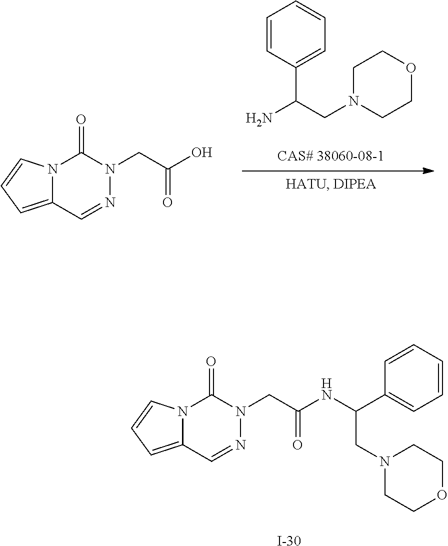

2-(4-Oxopyrrolo[1,2-d][1,2,4]triazin-3(4H)yl)acetic acid (0.08 g, 0.41 mmol) was dissolved in a solution of DMF (6 mL), then HATU (0.315 g, 0.83 mmol) was added thereto, and the reaction mixture was stirred at room temperature for 30 minutes. DIPEA (0.134 g, 1.03 mmol) and 2-morpholin-4-yl-1-phenylethylamine (CAS #38060 August 1, 0.094 g, 0.45 mmol) were sequentially added thereto and stirred at room temperature overnight. The reaction mixture was diluted with water and extracted with ethyl acetate. The combined organic phases were washed with brine, dried over anhydrous Na₂SO₄, filtered, and concentrated under vacuum. The remaining solid was purified by silica gel column chromatography (0 to 5% MeOH/DCM) to obtain compound I-30 (0.035 g, yield: 22%) as a brown foam. ¹H NMR (800 MHz, DMSO-d₆) δ 8.27 (s, 1H), 7.92 (t, J=5.7 Hz, 1H), 7.79 (d, J=1.9 Hz, 1H), 7.36-7.31 (m, 2H), 7.30-7.26 (m, 1H), 7.25-7.21 (m, 2H), 6.88-6.86 (m, 1H), 6.85-6.83 (m, 1H), 4.59 and 4.57 (ABq, J=16.3 Hz, 2H), 3.73-3.67 (m, 1H), 3.56-3.51 (m, 4H), 3.49 (d, J=6.8 Hz, 1H), 3.32 (d, J=6.1 Hz, 1H), 2.38-2.24 (m, 4H). ¹³C NMR (201 MHz, DMSO-d₆) δ 166.62, 144.52, 137.71, 132.35, 128.54 (2C), 128.02 (2C), 127.33, 125.59, 117.07, 115.94, 109.66, 67.92, 66.37 (2C) 52.86, 50.10 (2C), 40.27. HRMS (ESI) calculated for C₂₀H₂₄N₅O₃⁺ [M+H]⁺: 382.1874, found: 382.1875.

### Example 31: Preparation of (R)—N-(1-(4-fluorophenyl)ethyl-2-(4-oxopyrrolo[1,2-d][1,2,4]triazin-3(4H)yl)acetamide (compound I-31)

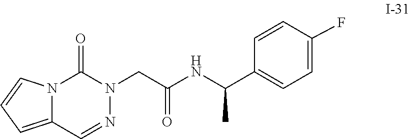 [SMILES: C[C@@H](NC(=O)Cn1ncc2cccn2c1=O)c1ccc(F)cc1]

The(S)-4-(1-aminoethyl) phenol in Example 7 was replaced with (R)-1-(4-fluorophenyl)ethylamine, and the rest of the required raw materials, reagents, and preparation methods were the same as those in Example 7, yielding compound I-31 (0.130 g, yield: 79%) as a white solid. ¹H NMR (800 MHZ, DMSO-d₆) δ 8.62 (d, J=7.9 Hz, 1H), 8.27 (s, 1H), 7.78 (d, J=2.2 Hz, 1H), 7.37-7.34 (m, 2H), 7.17-7.13 (m, 2H), 6.86 (dd, J=3.7, 1.4 Hz, 1H), 6.84 (t, J=3.3 Hz, 1H), 4.96-4.92 (m, 1H), 4.68 and 4.65 (ABq, J=16.4 Hz, 2H), 1.36 (d, J=7.0 Hz, 3H). ¹³C NMR (201 MHz, DMSO-d₆) δ 165.83, 161.04 (d, J=242.1 Hz), 144.61, 140.48, 132.33, 127.89 (2C, d, J=8.1 Hz), 125.62, 117.04, 115.90, 114.93 (2C, d, J=21.3 Hz), 109.61, 52.92, 47.51, 22.38. HRMS (ESI) calculated for C₁₆H₁₆FN₄O₂⁺ [M+H]⁺: 315.1252, found: 315.1252.

### Example 32: Preparation of (S)-2-(4-oxopyrrolo[1,2-d][1,2,4]triazin-3(4H)yl)-N-(1-(4-(trifluoromethyl)phenyl)ethyl)acetamide (compound I-32)

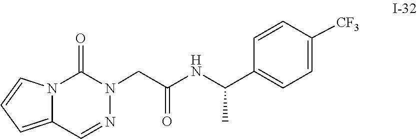 [SMILES: C[C@H](NC(=O)Cn1ncc2cccn2c1=O)c1ccc(C(F)(F)F)cc1]

The(S)-4-(1-aminoethyl) phenol in Example 7 was replaced with (S)-1-[4-(trifluoromethyl)phenyl]ethylamine hydrochloride, and the rest of the required raw materials, reagents, and preparation methods were the same as those in Example 7, yielding compound I-32 (0.105 g, yield: 79%) as a white solid. ¹H NMR (800 MHZ, DMSO-d₆) δ 8.75 (d, J=7.7 Hz, 1H), 8.28 (s, 1H), 7.79 (d, J=2.9 Hz, 1H), 7.70 (d, J=8.3 Hz, 2H), 7.55 (d, J=8.4 Hz, 2H), 6.86 (dd, J=3.7, 1.4 Hz, 1H), 6.85-6.83 (m, 1H), 5.03-4.98 (m, 1H), 4.70 (s, 2H), 1.39 (d, J=7.0 Hz, 3H). ¹³C NMR (201 MHz, DMSO-d₆) δ 166.10, 149.25, 144.61, 132.37, 127.41 (q, J=31.5 Hz), 126.74 (2C), 125.62, 125.20 (2C, q, J=3.7 Hz), 124.33 (q, J=271.8 Hz), 117.05, 115.91, 109.64, 52.93, 48.07, 22.16. HRMS (ESI) calculated for C₁₇H₁₆F₃N₄O₂⁺ [M+H]⁺: 365.1220, found: 365.1219.

### Example 33: Preparation of (S)—N-(1-(4-cyanophenylethyl)-2-(4-oxopyrrolo[1,2-d][1,2,4]triazin-3(4H)yl)acetamide (compound I-33)

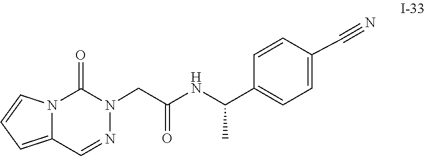 [SMILES: C[C@H](NC(=O)Cn1ncc2cccn2c1=O)c1ccc(C#N)cc1]

The(S)-4-(1-aminoethyl) phenol in Example 7 was replaced with (S)-4-(1-aminoethyl)benzyl cyanide hydrochloride, and the rest of the required raw materials, reagents, and preparation methods were the same as those in Example 7, yielding compound I-33 (0.060 g, yield: 72%) as a white solid. ¹H NMR (800 MHZ, DMSO-d₆) δ 8.75 (d, J=7.7 Hz, 1H), 8.28 (s, 1H), 7.82-7.80 (m, 2H), 7.78 (d, J=2.3 Hz, 1H), 7.53-7.51 (m, 2H), 6.86 (dd, J=3.7, 1.4 Hz, 1H), 6.85-6.83 (m, 1H), 5.01-4.96 (m, 1H), 4.70 (s, 2H), 1.38 (d, J=7.0 Hz, 3H). ¹³C NMR (201 MHz, DMSO-d₆) δ 166.17, 150.20, 144.61, 132.38, 132.33 (2C), 126.95 (2C), 125.61, 118.88, 117.06, 115.92, 109.66, 109.52, 52.95, 48.19, 22.03. HRMS (ESI) calculated for C₁₇H₁₆NO₂⁺ [M+H]⁺: 322.1299, found: 322.1297.

### Example 34: Preparation of (S)—N-(2-methyl-1-phenylpropyl)-2-(4-oxopyrrolo[1,2-d][1,2,4]triazin-3(4H)yl)acetamide (compound I-34)

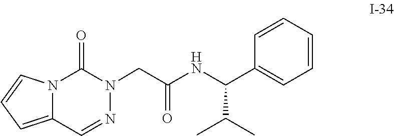 [SMILES: CC(C)[C@H](NC(=O)Cn1ncc2cccn2c1=O)c1ccccc1]

The(S)-4-(1-aminoethyl) phenol in Example 7 was replaced with (S)-2-methyl-1-phenylpropan-1-amine hydrochloride, and the rest of the required raw materials, reagents, and preparation methods were the same as those in Example 7, yielding compound I-34 (0.035 g, yield: 41%) as a white solid. ¹H NMR (800 MHZ, DMSO-d₆) δ 8.53 (d, J=9.0 Hz, 1H), 8.26 (s, 1H), 7.78 (d, J=4.7 Hz, 1H), 7.34-7.31 (m, 2H), 7.29-7.27 (m, 2H), 7.25-7.22 (m, 1H), 6.85 (dd, J=3.7, 1.4 Hz, 1H), 6.84 (t, J=3.4 Hz, 1H), 4.74 and 4.65 (ABq, J=15.9 Hz, 2H), 4.56 (t, J=8.6 Hz, 1H), 1.98-1.93 (m, 1H), 0.91 (d, J=6.7 Hz, 3H), 0.73 (d, J=6.7 Hz, 3H). ¹³C NMR (201 MHz, DMSO-d₆) δ 166.02, 144.55, 142.33, 132.21, 128.08 (2C), 127.09 (2C), 126.71, 125.59, 117.00, 115.90, 109.57, 58.83, 52.85, 32.99, 19.77, 18.91. HRMS (ESI) calculated for C₁₈H₂₁N₄O₂⁺ [M+H]⁺: 325.1659, found: 325.1659.

### Example 35: Preparation of (S)—N-(2-(4-methylpiperazin)-1-phenylethyl)-2-(4-oxopyrrolo[1,2-d][1,2,4]triazin-3(4H)yl)acetamide (compound I-35)

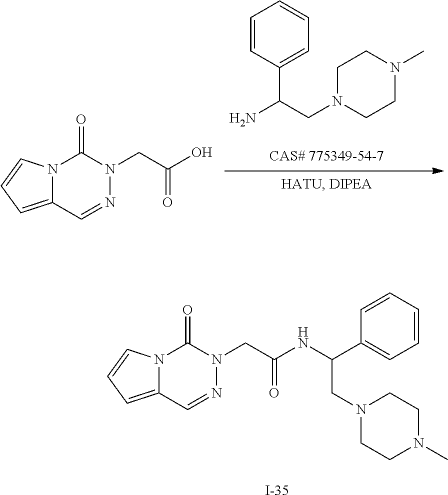

2-(4-Oxopyrrolo[1,2-d][1,2,4]triazin-3(4H)yl)acetic acid (0.05 g, 0.25 mmol) was dissolved in a solution of DMF (6 mL), then HATU (0.197 g, 0.51 mmol) was added thereto, and the reaction mixture was stirred at room temperature for 30 minutes. DIPEA (0.084 g, 0.64 mmol) and 2-(4-methylpiperazin-1-yl)-1-phenylethan-1-amine (CAS #775349-54-7, 0.063 g, 0.28 mmol) were sequentially added thereto and stirred at room temperature overnight. The reaction mixture was diluted with water and extracted with ethyl acetate. The combined organic phases were washed with brine, dried over anhydrous Na₂SO₄, filtered, and concentrated under vacuum. The remaining solid was purified by silica gel column chromatography (0 to 5% MeOH/DCM) to obtain compound I-35 (0.025 g, yield: 24%) as a brown foam. ¹H NMR (800 MHZ, DMSO-d₆) δ 8.19 (s, 1H), 7.86 (t, J=5.7 Hz, 1H), 7.79 (d, J=1.8 Hz, 1H), 7.33 (t, J=7.4 Hz, 2H), 7.28-7.26 (m, 1H), 7.24-7.22 (m, 2H), 6.88-6.86 (m, 1H), 6.86-6.84 (m, 1H), 4.59 and 4.57 (ABq, J=15.6 Hz, 2H), 3.69-3.65 (m, 1H), 3.53 (t, J=7.0 Hz, 1H), 3.35-3.31 (m, 1H), 2.50-2.18 (m, 8H), 2.16 (s, 3H). HRMS (ESI) calculated for C₂₁H₂₇N₆O₂⁺ [M+H]⁺: 395.2190, found: 395.2192.

### Example 36: Preparation of (S)—N-(1-cyclohexylethyl)-2-(4-oxopyrrolo[1,2-d][1,2,4]triazin-3(4H)yl)acetamide (compound I-36)

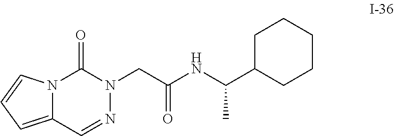 [SMILES: C[C@H](NC(=O)Cn1ncc2cccn2c1=O)C1CCCCC1]

The(S)-4-(1-aminoethyl) phenol in Example 7 was replaced with (S)-1-cyclohexylethylamine, and the rest of the required raw materials, reagents, and preparation methods were the same as those in Example 7, yielding compound I-36 (0.090 g, yield: 57%) as a light yellow solid. ¹H NMR (800 MHZ, DMSO-d₆) δ 8.27 (s, 1H), 7.91 (d, J=8.7 Hz, 1H), 7.80-7.77 (m, 1H), 6.87-6.83 (m, 2H), 4.66 and 4.58 (ABq, J=16.2 Hz, 2H), 3.65-3.59 (m, 1H), 1.76-1.64 (m, 4H), 1.62-1.54 (m, 1H), 1.33-1.22 (m, 1H), 1.20-1.05 (m, 3H), 1.00 (d, J=6.7 Hz, 3H), 0.97-0.82 (m, 2H). ¹³C NMR (201 MHz, DMSO-d₆) δ 166.13, 145.04, 132.70, 126.10, 117.47, 116.33, 109.99, 53.35, 49.35, 42.90, 29.26, 29.22, 26.48, 26.23, 26.21, 18.16. HRMS (ESI) calculated for C₁₆H₂₃N₄O₂⁺ [M+H]⁺: 303.1816, found: 303.1815.

### Example 37: Preparation of (S)—N-(1-(4-fluorophenyl)-2-methylpropyl)-2-(4-oxopyrrolo[1,2-d][1,2,4]triazin-3(4H)yl)acetamide (compound I-37)

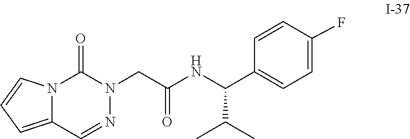 [SMILES: CC(C)[C@H](NC(=O)Cn1ncc2cccn2c1=O)c1ccc(F)cc1]

The(S)-4-(1-aminoethyl) phenol in Example 7 was replaced with (S)-1-(4-fluorophenyl)-2-methylpropan-1-amine hydrochloride, and the rest of the required raw materials, reagents, and preparation methods were the same as those in Example 7, yielding compound I-37 (0.100 g, yield: 80%) as a white solid. ¹H NMR (800 MHz, DMSO-d₆) δ 8.55 (d, J=8.9 Hz, 1H), 8.26 (s, 1H), 7.78 (d, J=2.3 Hz, 1H), 7.34-7.30 (m, 2H), 7.17-7.13 (m, 2H), 6.85 (dd, J=3.6, 1.4 Hz, 1H), 6.84-6.82 (m, 1H), 4.74 and 4.65 (ABq, J=16.4 Hz, 2H), 4.56 (t, J=8.6 Hz, 1H), 1.97-1.91 (m, 1H), 0.91 (d, J=6.7 Hz, 3H), 0.72 (d, J=6.7 Hz, 3H). ¹³C NMR (201 MHz, DMSO-d₆) δ 166.08, 161.03 (d, J=242.5 Hz), 144.56, 138.58, 132.25, 128.94 (2C, d, J=8.1 Hz), 125.60, 117.03, 115.92, 114.80 (2C, d, J=20.9 Hz), 109.61, 58.19, 52.87, 32.99, 19.68, 18.92. HRMS (ESI) calculated for C₁₈H₂₀FN₄O₂⁺ [M+H]⁺: 343.1565, found: 343.1564.

### Example 38: Preparation of (S)—N-(2-methyl-1-(4-trifluoromethyl)phenylpropyl)-2-(4-oxopyrrolo[1,2-d][1,2,4]triazin-3(4H)yl)acetamide (compound I-38)

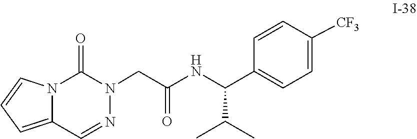 [SMILES: CC(C)[C@H](NC(=O)Cn1ncc2cccn2c1=O)c1ccc(C(F)(F)F)cc1]

The(S)-4-(1-aminoethyl) phenol in Example 7 was replaced with (S)-2-methyl-1-(4-(trifluoromethyl)phenyl) propan-1-amine, and the rest of the required raw materials, reagents, and preparation methods were the same as those in Example 7, yielding compound I-38 (0.110 g, yield: 77%) as a white solid. ¹H NMR (800 MHZ, DMSO-d₆) δ 8.66 (d, J=8.7 Hz, 1H), 8.26 (s, 1H), 7.79-7.76 (m, 1H), 7.70 (d, J=8.4 Hz, 2H), 7.52 (d, J=8.3 Hz, 2H), 6.86-6.84 (m, 1H), 6.84-6.82 (m, 1H), 4.76 and 4.68 (ABq, J=16.1 Hz, 2H), 4.65 (t, J=8.4 Hz, 1H), 2.04-1.95 (m, 1H), 0.93 (d, J=6.7 Hz, 3H), 0.74 (d, J=6.7 Hz, 3H). ¹³C NMR (201 MHz, DMSO-d₆) δ 166.83, 147.69, 145.02, 132.73, 128.36 (2C), 127.89 (q, J=31.5 Hz), 126.05, 125.48 (2C, q, J=3.7 Hz), 124.13 (q, J=264.9 Hz), 117.50, 116.39, 110.09, 59.06, 53.32, 33.20, 20.08, 19.22. HRMS (ESI) calculated for C₁₉H₂₀F₃N₄O₂⁺ [M+H]⁺: 393.1533, found: 393.1535.

### Example 39: Preparation of (S)—N-(1-(4-fluorophenyl)-2-methylpropyl)-2-(6-methyl-4-oxopyrrolo[1,2-d][1,2,4]triazin-3(4H)yl)acetamide (compound I-39)

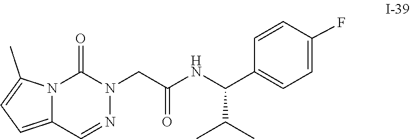 [SMILES: Cc1ccc2cnn(CC(=O)N[C@H](c3ccc(F)cc3)C(C)C)c(=O)n12]

2-(6-Methyl-4-oxopyrrolo[1,2-d][1,2,4]triazin-3(4H)yl)acetic acid (0.080 g, 0.39 mmol) was dissolved in a solution of DMF (5 mL), then HATU (0.295 g, 0.78 mmol) was added thereto, and the reaction mixture was stirred at room temperature for 20 minutes. DIPEA (0.125 g, 0.98 mmol) and (S)-1-(4-fluorophenyl)-2-methylpropan-1-amine hydrochloride (0.089 g, 0.62 mmol) were sequentially added thereto and stirred at room temperature overnight. The preparation method was the same as that in Example 26, yielding compound I-39 (0.110 g, yield: 79%) as a white solid. ¹H NMR (800 MHZ, DMSO-d₆) δ 8.50 (d, J=8.9 Hz, 1H), 8.06 (s, 1H), 7.35-7.29 (m, 2H), 7.17-7.12 (m, 2H), 6.66 (d, J=3.6 Hz, 1H), 6.50 (d, J=2.6 Hz, 1H), 4.64 (d, J=16.3 Hz, 1H), 4.58-4.54 (m, 2H), 2.67 (s, 3H), 1.96-1.88 (m, 1H), 0.91 (d, J=6.6 Hz, 3H), 0.72 (d, J=6.8 Hz, 3H). ¹³C NMR (201 MHz, DMSO-d₆) δ 166.25, 161.02 (d, J=242.5 Hz), 146.18, 138.63 (d, J=2.9 Hz), 132.57, 130.73, 128.93 (2C, d, J=8.1 Hz), 125.70, 115.43, 114.78 (2C, d, J=21.3 Hz), 108.28, 58.16, 52.57, 33.00, 19.67, 18.91, 14.67. HRMS (ESI) calculated for C₁₉H₂₂FN₄O₂⁺ [M+H]⁺: 357.1721, found: 357.1722.

### Example 40: Preparation of (S)—N-(2-methyl-1-(4-trifluoromethyl)phenylpropyl)-2-(6-methyl-4-oxopyrrolo[1,2-d][1,2,4]triazin-3(4H)yl)acetamide (compound I-40)

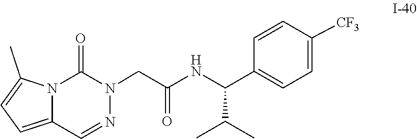 [SMILES: Cc1ccc2cnn(CC(=O)N[C@H](c3ccc(C(F)(F)F)cc3)C(C)C)c(=O)n12]

The(S)-1-(4-fluorophenyl)-2-methylpropan-1-amine hydrochloride in Example I-39 was replaced with (S)-2-methyl-1-(4-(trifluoromethyl)phenyl) propan-1-amine, and the rest of the required raw materials, reagents, and preparation methods were the same as those in Example 26, yielding compound I-40 (0.120 g, yield: 76%) as a white solid. ¹H NMR (800 MHz, DMSO-d₆) δ 8.61 (d, J=8.7 Hz, 1H), 8.06 (s, 1H), 7.70 (d, J=8.4 Hz, 2H), 7.52 (d, J=8.3 Hz, 2H), 6.66 (d, J=3.6 Hz, 1H), 6.50 (dd, J=3.7, 1.0 Hz, 1H), 4.68-4.63 (m, 2H), 4.59 (d, J=16.3 Hz, 1H), 2.67 (s, 3H), 2.02-1.97 (m, 1H), 0.92 (d, J=6.6 Hz, 3H), 0.74 (d, J=6.7 Hz, 3H). ¹³C NMR (201 MHz, DMSO-d₆) δ 166.54, 147.29, 146.18, 132.60, 130.74, 129.02 (q, J=35.1 Hz) 127.91 (2C), 125.70, 124.99 (2C, q, J=8.2 Hz), 124.33 (q, J=271.7 Hz), 115.44, 108.30, 58.58, 52.58, 32.74, 19.61, 18.75, 14.66. HRMS (ESI) calculated for C₂₀H₂₂F₃N₄O₂⁺ [M+H]⁺: 407.1689, found: 407.1690.

### Example 41: Preparation of (S)-2 (6-methyl-4-oxopyrrolo[1,2-d][1,2,4]triazin-3(4H)yl)-N-(1-phenylpropyl)acetamide (compound I-41)

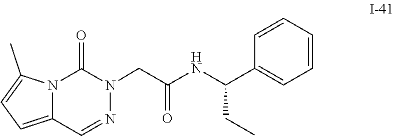 [SMILES: CC[C@H](NC(=O)Cn1ncc2ccc(C)n2c1=O)c1ccccc1]

The(S)-1-(1-naphthyl)ethylamine in Example 26 was replaced with (S)-1-phenylpropylamine, and the rest of the required raw materials, reagents, and preparation methods were the same as those in Example 26, yielding compound I-41 (0.05 g, yield: 31%) as a white solid. ¹H NMR (800 MHZ, DMSO-d₆) δ 8.51 (d, J=8.4 Hz, 1H), 8.07 (s, 1H), 7.34-7.28 (m, 4H), 7.25-7.21 (m, 1H), 6.67 (d, J=3.6 Hz, 1H), 6.51 (dd, J=3.7, 1.1 Hz, 1H), 4.73-4.69 (m, 1H), 4.62 and 4.57 (ABq, J=16.2 Hz, 2H), 2.68 (d, J=1.0 Hz, 3H), 1.73-1.66 (m, 2H), 0.85 (t, J=7.3 Hz, 3H). ¹³C NMR (201 MHz, DMSO-d₆) δ 166.29, 146.21, 143.38, 132.61, 130.73, 128.20 (2C), 126.70, 126.44 (2C), 125.73, 115.41, 108.25, 54.28, 52.65, 29.25, 14.67, 10.97. HRMS (ESI) calculated for C₁₈H₂₁N₄O₂⁺ [M+H]⁺: 325.1659, found: 325.1659.

### Example 42: Preparation of (S)-2 (6-methyl-4-oxopyrrolo[1,2-d][1,2,4]triazin-3(4H)yl)-N-(1,2,3,4-tetrahydronaphthalen-1-yl)acetamide (compound I-42)

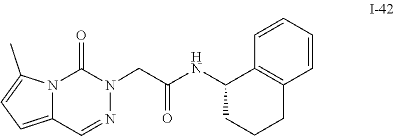 [SMILES: Cc1ccc2cnn(CC(=O)N[C@H]3CCCc4ccccc43)c(=O)n12]

The(S)-1-(1-naphthyl)ethylamine in Example 26 was replaced with (S)-1,2,3,4-tetrahydro-1-naphthylamine, and the rest of the required raw materials, reagents, and preparation methods were the same as those in Example 26, yielding compound I-42 (0.085 g, yield: 63%) as a white solid. ¹H NMR (800 MHZ, DMSO-d₆) δ 8.51 (d, J=8.7 Hz, 1H), 8.10 (s, 1H), 7.22-7.19 (m, 1H), 7.17-7.14 (m, 2H), 7.10-7.08 (m, 1H), 6.67 (d, J=3.6 Hz, 1H), 6.51 (dd, J=3.7, 1.0 Hz, 1H), 5.02-4.97 (m, 1H), 4.60 and 4.57 (ABq, J=16.4 Hz, 2H), 2.78-2.68 (m, 5H), 1.92-1.84 (m, 2H), 1.77-1.66 (m, 2H). ¹³C NMR (201 MHZ, DMSO-d₆) δ 166.33, 146.25, 137.24, 137.04, 132.70, 130.74, 128.71, 128.12, 126.76, 125.83, 125.80, 115.39, 108.23, 52.87, 46.72, 29.82, 28.74, 20.09, 14.70. HRMS (ESI) calculated for C₁₉H₂₁N₄O₂⁺ [M+H]⁺: 337.1659, found: 337.1657.

### Example 43: Preparation of (S)-2 (6-methyl-4-oxopyrrolo[1,2-d][1,2,4]triazin-3(4H)yl)-N-(1-(pyridin-3-yl)ethyl)acetamide (compound I-43)

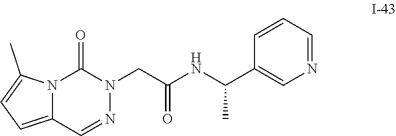 [SMILES: Cc1ccc2cnn(CC(=O)N[C@@H](C)c3cccnc3)c(=O)n12]

The(S)-1-(1-naphthyl)ethylamine in Example 26 was replaced with (S)-1-(pyridin-3-yl)ethylamine, and the rest of the required raw materials, reagents, and preparation methods were the same as those in Example 26, yielding compound I-43 (0.065 g, yield: 52%) as a white solid. ¹H NMR (800 MHZ, DMSO-d₆) δ 8.67 (d, J=7.7 Hz, 1H), 8.54 (d, J=2.3 Hz, 1H), 8.45 (dd, J=4.7, 1.6 Hz, 1H), 8.08 (s, 1H), 7.73-7.71 (m, 1H), 7.37-7.35 (m, 1H), 6.67 (d, J=3.6 Hz, 1H), 6.51 (dd, J=3.7, 1.1 Hz, 1H), 5.00-4.96 (m, 1H), 4.60 (s, 2H), 2.69 (d, J=0.9 Hz, 3H), 1.41 (d, J=7.0 Hz, 3H). ¹³C NMR (201 MHz, DMSO-d₆) δ 166.26, 147.98, 147.75, 146.23, 139.57, 133.59, 132.69, 130.76, 125.73, 123.40, 115.43, 108.32, 52.75, 46.23, 22.01, 14.67. HRMS (ESI) calculated for C₁₆H₁₈N₅O₂⁺ [M+H]⁺: 312.1455, found: 312.1454.

### Example 44: Preparation of N-(2-(dimethylamino)-1-phenylethyl)-2-(6-methyl-4-oxopyrrolo[1,2-d][1,2,4]triazin-3(4H)yl)acetamide (compound I-44)

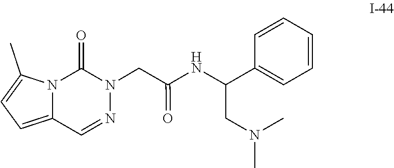 [SMILES: Cc1ccc2cnn(CC(=O)NC(CN(C)C)c3ccccc3)c(=O)n12]

The(S)-1-(1-naphthyl)ethylamine in Example 26 was replaced with N1,N1-dimethyl-2-phenylethane-1,2-diamine, and the rest of the required raw materials, reagents, and preparation methods were the same as those in Example 26, yielding compound I-44 (0.055 g, yield: 40%) as a brown foamy solid. ¹H NMR (800 MHZ, DMSO-d₆) δ 8.56 (d, J=5.3 Hz, 1H), 8.07 (s, 1H), 7.33 (d, J=4.4 Hz, 4H), 7.26-7.23 (m, 1H), 6.67 (d, J=3.7 Hz, 1H), 6.51 (dd, J=3.7, 1.0 Hz, 1H), 4.96 (s, 1H), 4.61 and 4.58 (ABq, J=16.2 Hz, 2H), 2.68 (s, 4H), 2.21 (brs, 6H), 1.26-1.22 (m, 1H). ¹³C NMR (201 MHz, DMSO-d₆) δ 166.76, 149.59, 146.68, 133.09, 131.22, 128.68 (2C), 127.40, 127.15 (2C), 126.16, 115.92, 108.78, 64.66, 53.08, 51.23, 45.48 (2C), 15.14. HRMS (ESI) calculated for C₁₉H₂₄N₅O₂⁺ [M+H]⁺: 354.1925, found: 354.1927.

### Example 45: Preparation of (R)—N-(1-(4-fluorophenyl)ethyl-2-(6-methyl-4-oxopyrrolo[1,2-d][1,2,4]triazin-3(4H)yl)acetamide (compound I-45)

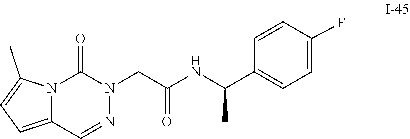 [SMILES: Cc1ccc2cnn(CC(=O)N[C@H](C)c3ccc(F)cc3)c(=O)n12]

The(S)-1-(1-naphthyl)ethylamine in Example 26 was replaced with (R)-1-(4-fluorophenyl)ethylamine, and the rest of the required raw materials, reagents, and preparation methods were the same as those in Example 26, yielding compound I-45 (0.040 g, yield: 18%) as a white solid. ¹H NMR (800 MHZ, DMSO-d₆) δ 8.59 (d, J=7.9 Hz, 1H), 8.08 (s, 1H), 7.38-7.34 (m, 2H), 7.17-7.13 (m, 2H), 6.67 (d, J=3.6 Hz, 1H), 6.50 (d, J=2.6 Hz, 1H), 4.96-4.92 (m, 1H), 4.58 and 4.55 (ABq, J=16.0 Hz, 2H), 2.69 (s, 3H), 1.36 (d, J=6.9 Hz, 3H). ¹³C NMR (201 MHz, DMSO-d₆) δ 166.02, 161.03 (d, J=242.1 Hz), 146.23, 140.54 (d, J=3.3 Hz), 132.65, 130.75, 127.90 (d, J=8.1 Hz, 2C), 125.73, 115.43, 114.91 (2C, d, J=20.9 Hz), 108.29, 52.69, 47.48, 22.43, 14.67. HRMS (ESI) calculated for C₁₇H₁₈FN₄O₂⁺ [M+H]⁺: 329.1408, found: 329.1407.

### Example 46: Preparation of (S)-2-(6-methyl-4-oxopyrrolo[1,2-d][1,2,4]triazin-3(4H)yl)-N-(1-(4-(trifluoromethyl)phenyl)ethyl)acetamide (compound I-46)

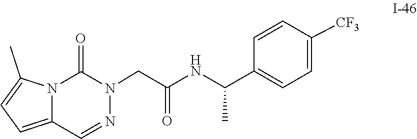 [SMILES: Cc1ccc2cnn(CC(=O)N[C@@H](C)c3ccc(C(F)(F)F)cc3)c(=O)n12]

The(S)-1-(1-naphthyl)ethylamine in Example 26 was replaced with (S)-1-[4-(trifluoromethyl)phenyl]ethylamine hydrochloride, and the rest of the required raw materials, reagents, and preparation methods were the same as those in Example 26, yielding compound I-46 (0.092 g, yield: 71%) as a white solid. ¹H NMR (800 MHZ, DMSO-d₆) δ 8.71 (d, J=7.6 Hz, 1H), 8.08 (s, 1H), 7.69 (d, J=8.5 Hz, 2H), 7.55 (d, J=8.3 Hz, 2H), 6.67 (d, J=3.6 Hz, 1H), 6.50 (dd, J=3.7, 1.0 Hz, 1H), 5.03-4.98 (m, 1H), 4.61 (s, 2H), 2.69 (s, 3H), 1.39 (d, J=7.2 Hz, 3H). ¹³C NMR (201 MHz, DMSO-d₆) δ 166.30, 149.32, 146.23, 132.69, 130.77, 127.32 (q, J=31.5 Hz), 126.75 (2C), 125.73, 125.19 (2C, q, J=3.7 Hz), 124.34 (q, J=271.8 Hz), 115.44, 108.32, 52.71, 48.04, 22.20, 14.66. HRMS (ESI) calculated for C₁₈H₁₈F₃N₄O₂⁺ [M+H]⁺: 379.1376, found: 379.1377.

### Bioassay Example 1: Determination of the Agonistic Activity of the Compounds of the Present Disclosure on GPR139

The agonistic activity of the compounds of the present disclosure on GPR139 was determined using a calcium flux detection assay (Molecular Devices). The specific operation method is as follows:
(1) Cell culture (CHO-K1 cell line, cell culture medium: Ham's F12+10% FBS+1% PS (Penicillin-Streptomycin)).
(2) In a 10 cm petri dish, cells were washed with 2 mL of PBS buffer (HyClone, Cat #SH30028.02), then the buffer was removed, and then 1 mL of trypsin (Thermo, Cat #25300054) was added to digest the cells for 2 minutes. The digestion was stopped by the addition of 2 mL of cell culture medium, and the mixture was mixed by pipetting and centrifuged at 1000 rpm for 3 minutes.
(3) The cells were resuspended and adjusted to a density of 3×10⁶ cells/mL, and 1 mL of the cell suspension was added to a 10 cm petri dish (10 mL of cell culture medium) and cultured overnight.
(4) When the cell density reached 60%, transfection was carried out. The cells were washed with fresh medium 1 hour before transfection. The GPR139 plasmid and TransIT-2020 were each added to Opti-MEM (transfection ratio: 1 μg GPR139 plasmid+16 μL TransIT-2020+1000 μL Opti-MEM), mixed by pipetting, and incubated at room temperature for 20 minutes. The mixture was then added dropwise to the petri dish containing the cells, gently mixed, and the transfection was carried out for 16 to 20 hours.
(5) After trypsinization, the cells were resuspended in 17 mL of cell culture medium and seeded into a black-bottomed transparent 384-well plate (Greiner, Cat #781091), with 40 μL of cell suspension added to each well, resulting in 15,000 to 20,000 cells per well. The plate was incubated at 37° C. with 5% CO₂ for 16 hours.
(6) Preparation of reagents: The compounds to be tested were prepared as a 10 mM stock solution in DMSO, then diluted to 30 μM in a 0.1% BSA (bovine serum albumin) solution, and further subjected to 3-fold serial dilution to obtain 16 concentration points. The agonist positive control TAK-041 and the blank control DMSO were both initially prepared at 30 μM and subjected to 16 concentration points.
(7) Using a multi-channel pipette, the compounds to be tested with different concentrations were transferred to a 384-well plate (Greiner, Cat #784201), 20 μL per well. The plate was centrifuged at 1000 rpm for 1 minute.
(8) The cell culture medium was removed from the black 384-well plate, and 20 μL of 1× dye solution (FLIPR Calcium 6 Assay Explorer Kit, Cat #R8190, containing 2.5 mM probenecid) was added to each well. The plate was then incubated at 37° C. for 60 minutes and equilibrated at room temperature for 15 minutes.
(9) Using a FLIPR (Molecular Devices), the compounds to be tested with different concentrations were transferred to the black 384-well plate, 10 μL per well. The FLIPR was used to read the calcium flux signal (maximum excitation light: 470 to 495 nm, maximum emission light: 515 to 575 nm).
(10) Data were analyzed using GraphPad Prism (version 8.0), with the data presented as mean±standard deviation. Concentration-response curves were fitted using nonlinear regression, and the EC₅₀ values were calculated.

Results: The agonistic activity of the compounds of the present disclosure on GPR139 is shown in Table 1.

TABLE 1

| Compound | EC₅₀ (nM) | Eₘₐₓ (%) |
|---|---|---|
| TAK-041 | 61 ± 2 | 100 |
| I-1 | 56 ± 13 | 101 |
| I-2 | 28 ± 4 | 112 |
| I-3 | 60 ± 1 | 114 |
| I-4 | 111 ± 4 | 99 |
| I-5 | 25 ± 2 | 105 |
| I-6 | 30 ± 6 | 111 |
| I-7 | NT | NT |
| I-8 | 45 ± 1 | 98 |
| I-9 | NT | NT |
| I-10 | NT | NT |
| I-11 | 19 ± 3 | 109 |
| I-12 | 21 ± 5 | 114 |
| I-13 | 24 ± 6 | 116 |
| I-14 | 13 ± 2 | 111 |
| I-15 | 75 ± 11 | 96 |
| I-16 | 23 ± 5 | 116 |
| I-17 | 95 ± 14 | 95 |
| I-18 | 93 ± 15 | 105 |
| I-19 | 34 ± 6 | 104 |
| I-20 | 19 ± 3 | 103 |
| I-21 | 96 ± 14 | 98 |
| I-22 | 56 ± 11 | 104 |
| I-23 | 23 ± 5 | 110 |
| I-24 | 33 ± 5 | 108 |
| I-25 | NT | NT |
| I-26 | 42 ± 1 | 110 |
| I-27 | 15 ± 3 | 108 |
| I-28 | NT | NT |
| I-29 | NT | NT |
| I-30 | NT | NT |
| I-31 | NT | NT |
| I-32 | 61 ± 4 | 105 |
| I-33 | NT | NT |
| I-34 | 57 ± 5 | 92 |
| I-35 | NT | NT |
| I-36 | NT | NT |
| I-37 | 120 | 92 |
| I-38 | 61 | 93 |
| I-39 | 26 | 94 |
| I-40 | 125 | 80 |
| I-41 | 22 ± 2 | 107 |
| I-42 | NT | NT |
| I-43 | NT | NT |
| I-44 | NT | NT |
| I-45 | NT | NT |
| I-46 | 68 ± 8 | 102 |

Note:
In the table above, Eₘₐₓ ± maximum effect of the compound of the present disclosure/maximum effect of TAK-041;
NT, not tested.

### Bioassay Example 2: Study on the Pharmacokinetics of Compounds in Mice In Vivo after Intraperitoneal Injection Administration

After a single-dose intraperitoneal injection administration of the compound in male mice in vivo, blood samples and brain tissue were collected at various time points. The concentrations of the compound in the mouse plasma and brain tissue were determined using LC-MS/MS, and the relevant pharmacokinetic parameters were calculated to investigate the pharmacokinetic characteristics and brain distribution of the compounds in mice in vivo.

### 2.1 Experimental Design

Thirty-six male C57 mice were randomly divided into 4 groups according to body weight, with 9 mice in each group. They were fasted for 12 to 14 hours without water restriction the day before administration and were given food 4 hours after administration. Compound solvent: 10% DMSO+10% solutol (polyethylene glycol-12 hydroxystearate)+80% normal saline.

| | | Administration information | | | | | |
|---|---|---|---|---|---|---|---|
| | | | | Adminis- | | | |
| | | | Adminis- | tration | Adminis- | | |
| | | | tration | concen- | tration | | Route of |
| | Quantity | Test | dose | tration | volume | Sample | adminis- |
| Group | Male | substance | (mg/kg) | (mg/mL) | (mL/kg) | collection | tration |
| G1 | 9 | TAK-041 | 10 | 1 | 10 | Plasma | Intraper- |
| G2 | 9 | I-1 | 10 | 1 | 10 | and brain | itoneal |
| G3 | 9 | I-5 | 10 | 1 | 10 | tissue | injection |
| G4 | 9 | I-32 | 10 | 1 | 10 | | |

### 2.2 Sample Collection

Before and after administration, blood (0.1 mL) was collected from the orbit under isoflurane anesthesia, placed in EDTA K2 centrifuge tubes, and placed on an ice bath. The samples were centrifuged at 5000 rpm for 10 minutes at 4° C., and the plasma was collected. IP plasma and brain tissue were collected at 0.5 hours, 2 hours, and 4 hours. All plasma samples were stored at −80° C. before analysis. The brain tissue was collected at 0.5 hours, 2 hours, and 4 hours. After the mice were bled and euthanized, the brain tissue was collected and cleaned, weighed accurately, and homogenized with 50% methanol in water at a ratio of 1:4. The homogenate samples were stored at −80° C. for analysis.

### 2.3 Data Processing

The data acquisition and control system software was Analyst 1.5.1 (Applied Biosystem). The sample peak integration mode of the spectrum was automatic integration; the ratio of the sample peak area to the internal standard peak area was used as an indicator to perform regression with the concentration of the sample. Regression mode: linear regression with a weight coefficient of 1/X². Pharmacokinetic parameters were analyzed using non-compartmental model analysis using WinNonlin Professional v6.3 (Pharsight, USA). Cₘₐₓ was the measured maximum plasma concentration, and the area under the plasma concentration-time curve (AUC_{(0→t)}) was calculated using the trapezoidal rule, and tₘₐₓ was the time at which the peak plasma concentration was reached after administration. Experimental data are expressed as “mean” (n=3).

### 2.4 Experimental Results

| | | TAK-041 | | I-1 | | I-5 | | I-32 | |
|---|---|---|---|---|---|---|---|---|---|
| Parameters | | Brain | Plasma | Brain | Plasma | Brain | Plasma | Brain | Plasma |
| T_{1/2} | h | 108 | NA | 0.32 | 0.70 | 0.90 | 0.98 | 0.53 | 0.52 |
| tₘₐₓ | h | 0.5 | 6.0 | 0.5 | 0.5 | 0.5 | 0.5 | 0.5 | 0.5 |
| Cₘₐₓ | ng · g⁻¹ | 6754 | 7057 | 713 | 700 | 1578 | 1844 | 1264 | 868 |
| AUC₀₋ₜ | ng · g⁻¹ | 34340 | 38478 | 734 | 806 | 1722 | 2094 | 1842 | 1244 |

| | | | | |
|---|---|---|---|---|
| Brain/plasma | 0.892 | 0.911 | 0.822 | 1.48 |

Note:
NA means that it is unable to be calculated.

### Bioassay Example 3: Behavioral Study on BALB/c Mice after Compound Administration

BALB/c mice were intraperitoneally injected with different doses of I-5, and the therapeutic effect of the developed compounds on the negative symptoms of schizophrenia was verified through social interaction experiments.

### 3.1 Experimental Subjects

The experimental subjects were 6-week-old male BALB/c wild-type mice, weighing approximately 22 grams, purchased from Shanghai Lingchang Biotechnology Co., Ltd. They were housed in SPF-grade breeding facilities, with 2 mice per cage, under a 12-hour light/dark cycle, with free access to food and water. All behavioral experiments in this project were completed during the light cycle. The experiment adhered to all relevant regulations and guidelines for animal welfare and ethics, and was supervised and inspected by the ethics committee and laboratory animal managers.

### 3.2 Behavioral System

The length, width, and height of the behavioral box used in this experiment were all 40 cm. During the experiment, 4 mice were tested in 4 behavioral boxes at the same time. In this experiment, Etho Vision XT was used for experimental setup and video recording.

### 3.3 Experimental Process

The mice were randomly divided into 6 groups, which were a negative control group; positive control groups: TAK-041 (3 mg/kg), TAK-041 (1 mg/kg); experimental groups: I-5 (3 mg/kg), I-5 (1 mg/kg), I-5 (0.3 mg/kg), ≥12 animals per group.

The mice were 5 weeks old when purchased. After one week of observation, the experiment was started when they were 6 weeks old and weighed about 22 grams. Before each experiment, the experimental mice were restricted from eating 18 hours in advance. On the day of the experiment, the mice were transferred to the behavioral laboratory to familiarize themselves with the environment 1 hour in advance. Subsequently, they were administered by intraperitoneal injection, and each mouse was returned to its original cage after injection. After 20 minutes, the experimental mice were placed in the behavioral box to acclimate for 10 minutes. Then, a stimulus mouse (from the same batch of BALB/c) was placed in a corner of the behavioral box, away from the experimental mouse. The two mice were allowed to explore freely for 10 minutes while their activity was monitored and recorded by a camera, and the data was archived.

The experimental data were analyzed. The social interaction time (including approaching, sniffing, grooming, chasing, attacking, etc.) was calculated for each experimental mouse over a 10-minute period, and the total social time of each mouse across different groups was analyzed by one-way analysis of variance. During the analysis, mice that did not engage in social interactions or had social interaction times of less than 10 seconds were excluded.

### 3.4 Experimental Results

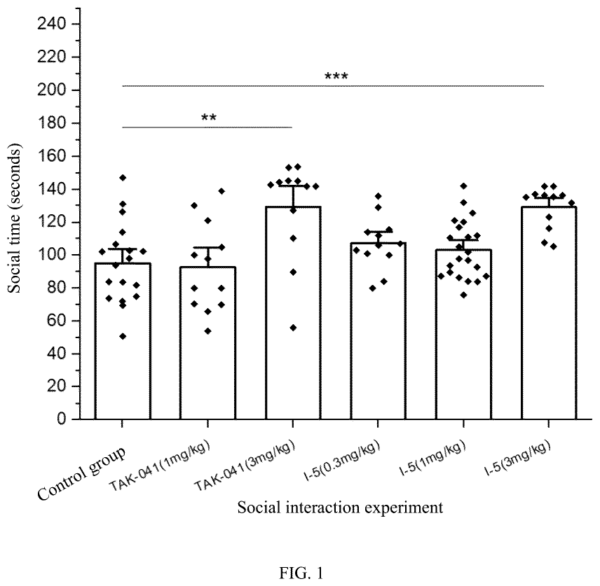

The experimental results are shown in FIG. 1 and the table below:

| | | | One-way |
|---|---|---|---|
| | | Social time | analysis of |
| Test compound | Dose | (seconds) | variance |
| Normal saline | — | 95 ± 24 | — |
| TAK-041 | 1 mg/kg | 93 ± 26 | No difference |
| | 3 mg/kg | 129 ± 28 | P < 0.01 |
| I-5 | 0.3 mg/kg | 107 ± 15 | No difference |
| | 1 mg/kg | 103 ± 17 | No difference |
| | 3 mg/kg | 129 ± 12 | P < 0.001 |

Experimental results show that compound I-5 has a significant improvement effect on social disorders in mice at a dose of 3 mg/kg.

1. A pyrrolotriazinone compound of formula I or a pharmaceutically acceptable salt thereof; 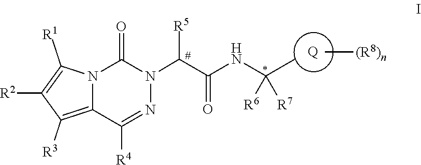
wherein
R¹, R², R³, and R⁴ are independently H, halogen, C₁₋₆ alkyl, C₃₋₆ cycloalkyl, —O—C₁₋₆ alkyl, C₁₋₆ alkyl substituted by 1, 2, or 3 R¹⁻¹, C₃₋₆ cycloalkyl substituted by 1, 2, or 3 R¹⁻², or —O—C₁₋₆ alkyl substituted by 1, 2, or 3 R¹⁻³,
R¹⁻¹, R¹⁻², and R¹⁻³ are independently halogen;
R⁵ is H or C₁₋₆ alkyl;
R⁶ and R⁷ are independently is H, C₁₋₆ alkyl, C₃₋₆ cycloalkyl, C₁₋₆ alkyl substituted by 1, 2, or 3 R⁶⁻¹, or C₃₋₆ cycloalkyl substituted by 1, 2, or 3 R⁶⁻²,
R⁷ is C₁₋₆ alkyl, C₃₋₆ cycloalkyl, C₁₋₆ alkyl substituted by 1, 2, or 3 R⁶⁻¹, or C₃₋₆ cycloalkyl substituted by 1, 2, or 3 R⁶⁻²;
R⁶⁻¹ is independently halogen, —O—C₁₋₆ alkyl, or —NR^{a}R^{b};
R⁶⁻² is independently halogen, —O—C₁₋₆ alkyl, or —NR^{a}R^{b},
R^{a} and R^{b} are independently C₁₋₆ alkyl;
or, R^{a} and R^{b}, together with the nitrogen atom to which they are attached, form a 5- to 6-membered heterocycloalkyl group or a 5- to 6-membered heterocycloalkyl group substituted by 1, 2, or 3 R^{a-1}; the 5- to 6-membered heterocycloalkyl group is independently a 5- to 6-membered heterocycloalkyl group with 1, 2, or 3 heteroatoms independently selected from 1, 2, or 3 kinds of N, O, and S;
R^{a-1} is independently C₁₋₆ alkyl;
Q is a C₆₋₁₀ aromatic ring, a 5- to 10-membered heteroaromatic ring, C₃₋₆ cycloalkyl, or 5- to 6-membered heterocycloalkyl; the 5- to 10-membered heteroaromatic ring is a 5- to 10-membered heteroaromatic ring with 1, 2, or 3 heteroatoms independently selected from 1, 2, or 3 kinds of N, O, and S;
n is 0, 1, 2, 3, 4, or 5;
R⁸ is independently halogen, —OH, —CN, —NH₂, C₁₋₆ alkyl, —O—C₁₋₆ alkyl, C₁₋₆ alkyl substituted by 1, 2, or 3 R⁸⁻¹, or —O—C₁₋₆ alkyl substituted by 1, 2, or 3 R⁸⁻²;
R⁸⁻¹ and R⁸⁻² are independently halogen;
or, R⁸ and R⁷ together form —(CH₂)ₘ—, m is 2 or 3;
carbon atoms with “*” and “#” are either achiral carbon atoms or chiral carbon atoms, and when they are chiral carbon atoms, they are independently in S configuration and/or R configuration.

2. The pyrrolotriazinone compound of formula I or the pharmaceutically acceptable salt thereof according to claim 1;
wherein
Q is a C₆₋₁₀ aromatic ring or a 5- to 10-membered heteroaromatic ring; the 5- to 10-membered heteroaromatic ring is a 5- to 10-membered heteroaromatic ring with 1, 2, or 3 heteroatoms independently selected from 1, 2, or 3 kinds of N, O, and S.

3. The pyrrolotriazinone compound of formula I or the pharmaceutically acceptable salt thereof according to claim 1, wherein

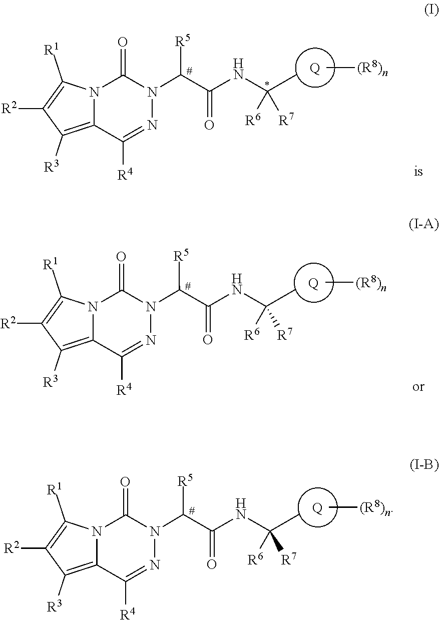

4. The pyrrolotriazinone compound of formula I or the pharmaceutically acceptable salt thereof according to claim 1, wherein it satisfies one or more of the following conditions:
(1) R¹, R², and R³ are independently H or C₁₋₆ alkyl;
(2) R⁴ is independently H;
(3) R⁵ is H;
(4) R⁶ is H;
(5) R⁷ is C₁₋₆ alkyl or C₁₋₆ alkyl substituted by 1, 2, or 3 R⁶⁻¹,
(6) R⁶⁻¹ is independently —NR^{a}R^{b},
(7) R^{a} and R^{b} are independently C₁₋₆ alkyl;
(8) n is 0 or 1; and
(9) R⁸ is halogen, —OH, —CN, C₁₋₆ alkyl, —O—C₁₋₆ alkyl, C₁₋₆ alkyl substituted by 1, 2, or 3 R⁸⁻¹, or —O—C₁₋₆ alkyl substituted by 1, 2, or 3 R⁸⁻²;
or, R⁸ and R⁷ together form —(CH₂)ₘ—, m is 2 or 3.

5. The pyrrolotriazinone compound of formula I or the pharmaceutically acceptable salt thereof according to claim 1, wherein it satisfies one or more of the following conditions:
(1) in R¹, R², R³, and R⁴, the C₁₋₆ alkyl is independently methyl, ethyl, n-propyl, isopropyl, n-butyl, isobutyl, or tert-butyl;
(2) in R¹⁻¹, R¹⁻², and R¹⁻³, the halogen is F, Cl, Br, or I;
(3) in R⁶ and R⁷, the “C₁₋₆ alkyl” in the C₁₋₆ alkyl and C₁₋₆ alkyl substituted by 1, 2, or 3 R⁶⁻¹ is independently methyl, ethyl, n-propyl, isopropyl, n-butyl, isobutyl, or tert-butyl;
(4) in R^{a} and R^{b}, the C₁₋₆ alkyl is independently methyl, ethyl, n-propyl, isopropyl, n-butyl, isobutyl, or tert-butyl;
(5) in Q, the C₆₋₁₀ aromatic ring is a benzene ring or a naphthalene ring;
(6) in Q, the 5- to 10-membered heteroaromatic ring is a 5- to 6-membered heteroaromatic ring;
(7) in R⁸, the halogen is independently F, Cl, Br, or I;
(8) in R⁸, the C₁₋₆ alkyl is independently methyl, ethyl, n-propyl, isopropyl, n-butyl, isobutyl, or tert-butyl;
(9) in R⁸, the “—O—C₁₋₆ alkyl” in the —O—C₁₋₆ alkyl and —O—C₁₋₆ alkyl substituted by 1, 2, or 3 R⁸⁻² is independently methoxy, ethoxy, n-propoxy, isopropoxy, n-butoxy, isobutoxy, or tert-butoxy;
(10) in R⁸⁻¹ and R⁸⁻², the halogen is independently F, Cl, Br, or I;
(11) when R⁸ and R⁷ together form —(CH₂)ₘ—, m is 3;
(12) when R^{a} and R^{b}, together with the nitrogen atom to which they are attached, form a 5- to 6-membered heterocycloalkyl group or a 5- to 6-membered heterocycloalkyl group substituted by 1, 2, or 3 R^{a-1}, the “5- to 6-membered heterocycloalkyl group” in the 5- to 6-membered heterocycloalkyl group or 5- to 6-membered heterocycloalkyl group substituted by 1, 2, or 3 R^{a-1} is independently a 5- to 6-membered heterocycloalkyl group with 1 or 2 heteroatoms independently selected from N and O;
(13) in R^{a-1}, the C₁₋₆ alkyl is independently methyl, ethyl, n-propyl, isopropyl, n-butyl, isobutyl, or tert-butyl, and
(14) in Q, the C₃₋₆ cycloalkyl is cyclopropyl, cyclobutyl, cyclopentyl, or cyclohexyl.

6. The pyrrolotriazinone compound of formula I or the pharmaceutically acceptable salt thereof according to claim 5, wherein it satisfies one or more of the following conditions:
(1) in R¹, R², R³, and R⁴, the C₁₋₆ alkyl is methyl;
(2) in R⁶ and R⁷, the “C₁₋₆ alkyl” in the C₁₋₆ alkyl and C₁₋₆ alkyl substituted by 1, 2, or 3 R⁶⁻¹ is independently methyl or ethyl;
(3) in R^{a} and R^{b}, the C₁₋₆ alkyl is methyl;
(4) in Q, the C₆₋₁₀ aromatic ring is a benzene ring;
(5) in Q, the 5- to 10-membered heteroaromatic ring is a pyridine ring;
(6) in R⁸, the halogen is F, Cl, or Br;
(7) in R⁸, the C₁₋₆ alkyl is methyl;
(8) in R⁸, the “—O—C₁₋₆ alkyl” in the —O—C₁₋₆ alkyl and —O—C₁₋₆ alkyl substituted by 1, 2, or 3 R⁸⁻² is methoxy;
(9) in R⁸⁻¹ and R⁸⁻², the halogen is F;
(10) when R^{a} and R^{b}, together with the nitrogen atom to which they are attached, form a 5- to 6-membered heterocycloalkyl group or a 5- to 6-membered heterocycloalkyl group substituted by 1, 2, or 3 R^{a-1}, the 5- to 6-membered heterocycloalkyl group is 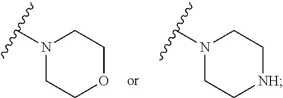 [SMILES: *N1CCOCC1 ; *N1CCNCC1]
(11) in R^{a-1}, the C₁₋₆ alkyl is methyl; and
(12) in Q, the C₃₋₆ cycloalkyl is cyclohexyl.

7. The pyrrolotriazinone compound of formula I or the pharmaceutically acceptable salt thereof according to claim 1, wherein it satisfies one or more of the following conditions:
(1) when Q is a benzene ring, then 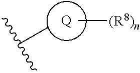
is 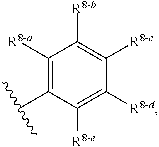
R^{8-a}, R^{8-b}, R^{8-c}, and R^{8-d} are independently H, halogen, —OH, —CN, —NH₂, C₁₋₆ alkyl, —O—C₁₋₆ alkyl, C₁₋₆ alkyl substituted by 1, 2, or 3 R⁸⁻¹, or —O—C₁₋₆ alkyl substituted by 1, 2, or 3 R⁸⁻²,
R^{8-e} is H, halogen, —OH, —CN, —NH₂, C₁₋₆ alkyl, —O—C₁₋₆ alkyl, C₁₋₆ alkyl substituted by 1, 2, or 3 R⁸⁻¹, or —O—C₁₋₆ alkyl substituted by 1, 2, or 3 R⁸⁻²,
or, R^{8-e} and R⁷ together form —(CH₂)ₘ—, m is 2 or 3;
(2) 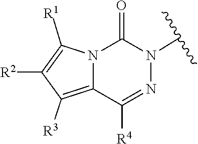
is 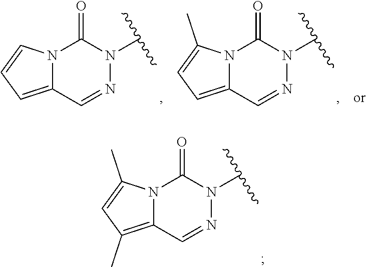
(3) R⁶ is H;
(4) R⁷ is —CH₃, —CH₂CH₃,

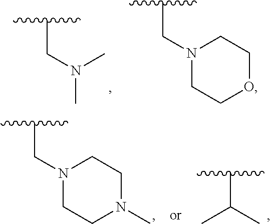

8. The pyrrolotriazinone compound of formula I or the pharmaceutically acceptable salt thereof according to claim 1, wherein R⁸ is independently F, Cl, Br, —OH, —CH₃, —OCH₃, —OCF₃, —CF₃, or CN.

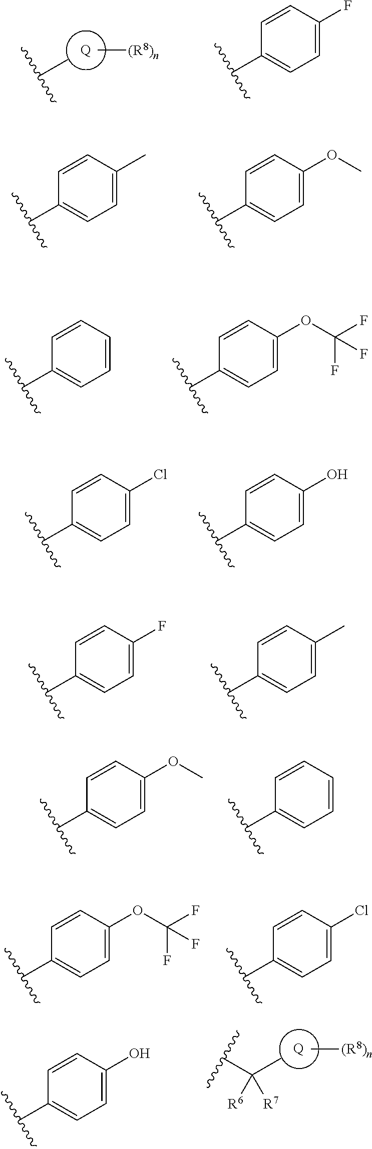

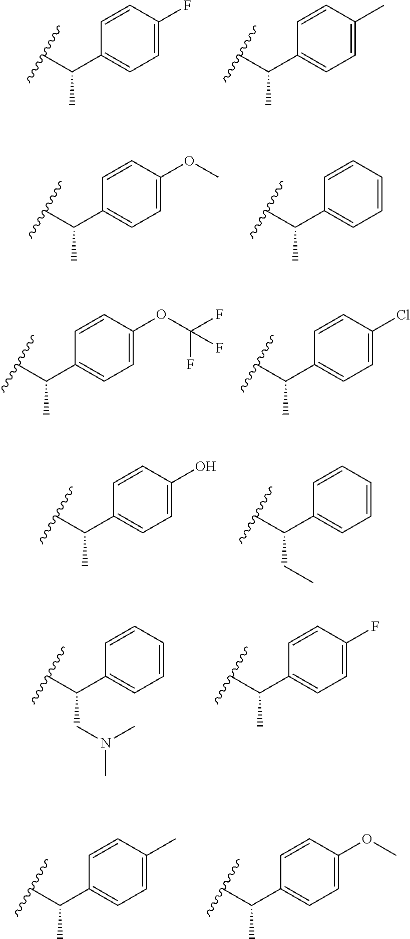

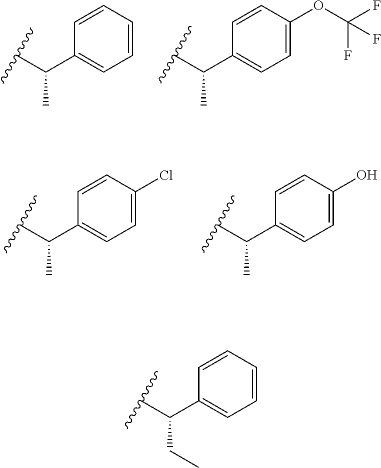

9. The pyrrolotriazinone compound of formula I or the pharmaceutically acceptable salt thereof according to claim 1, wherein the pyrrolotriazinone compound of formula I is selected from any one of the following compounds:

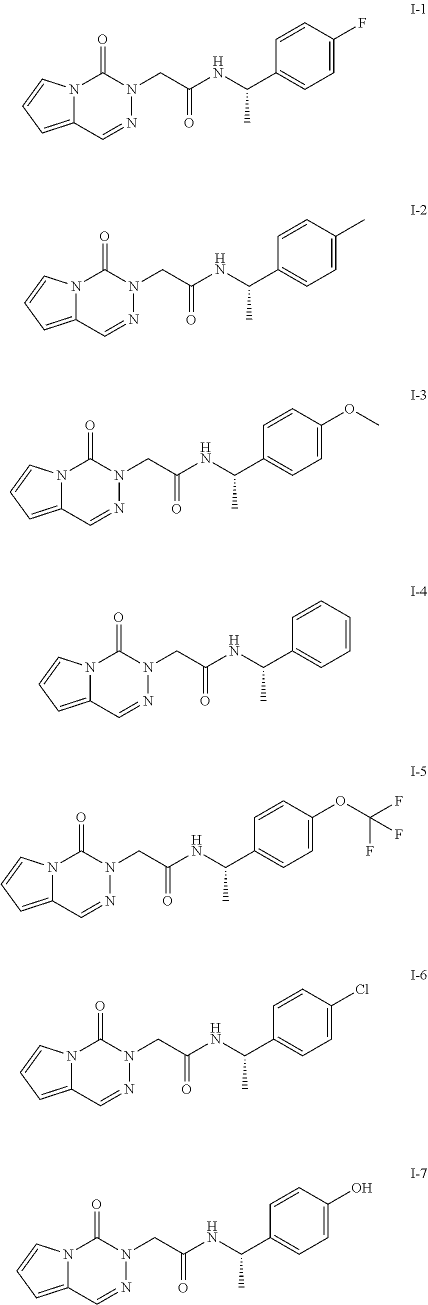

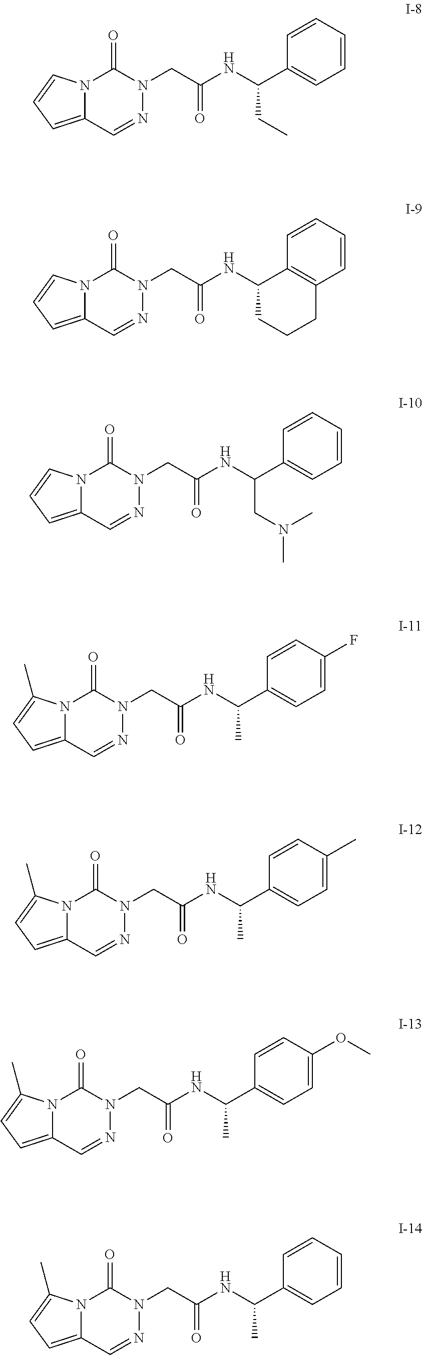

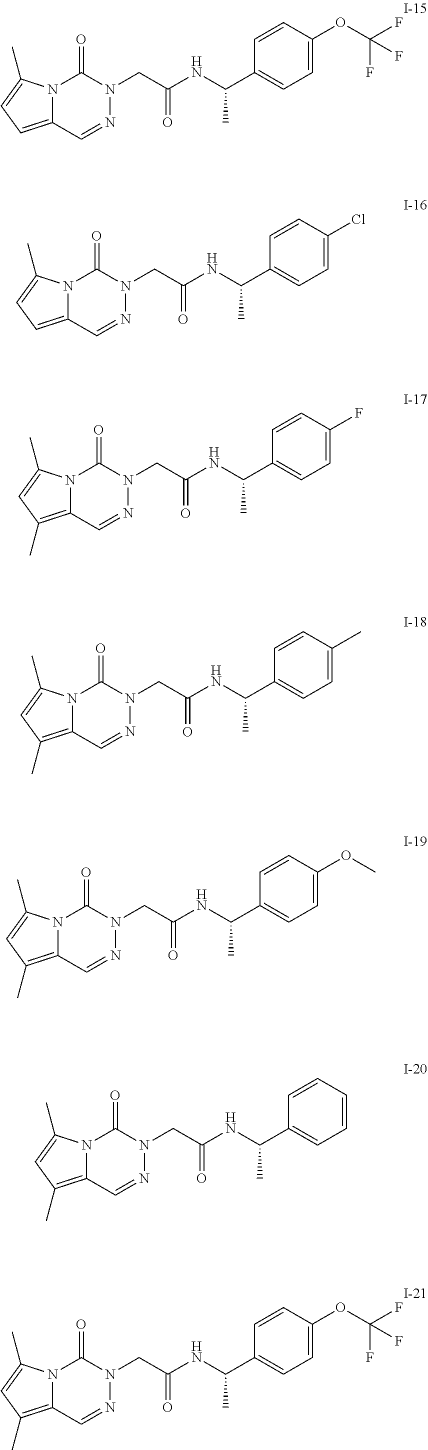

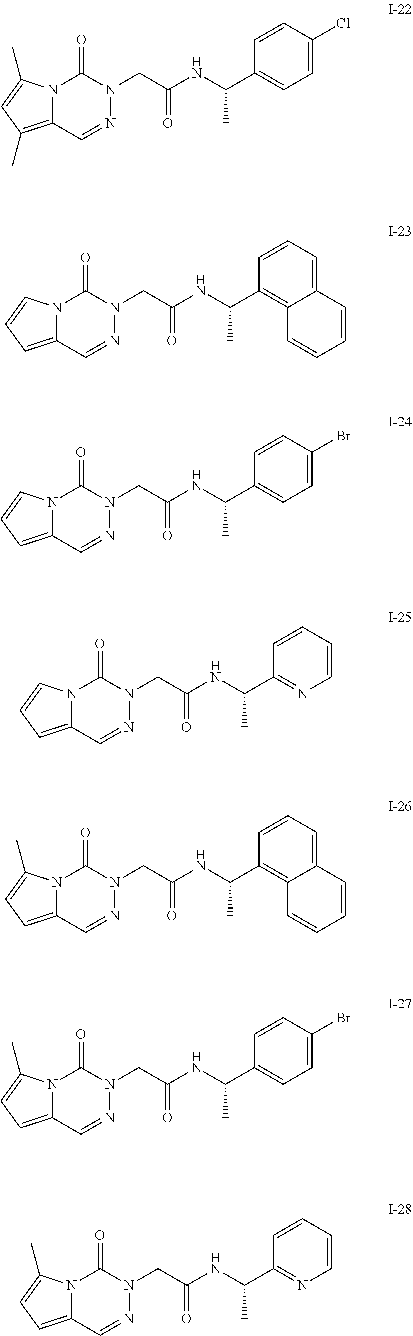

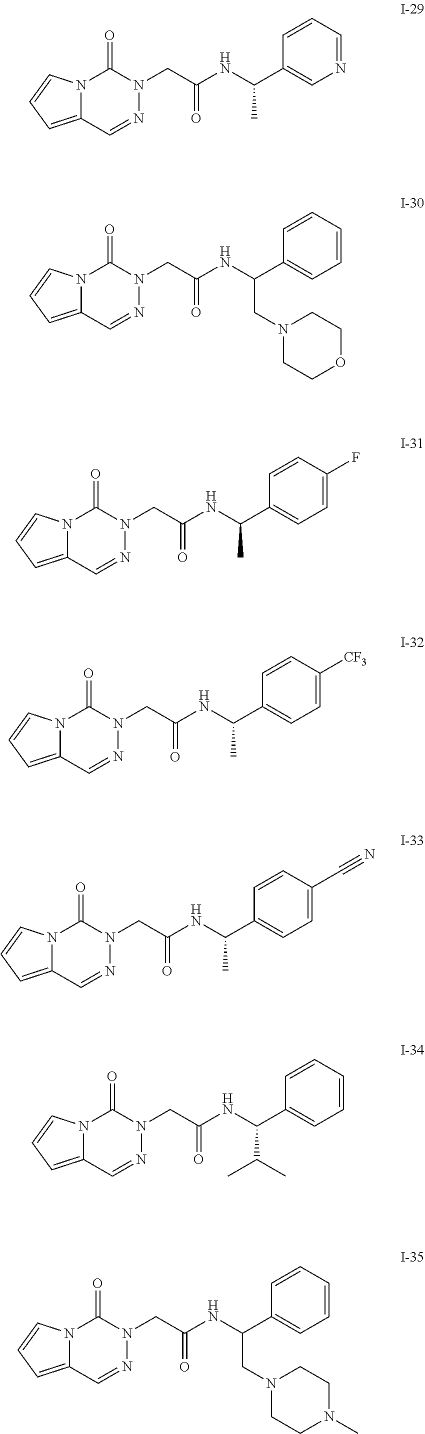

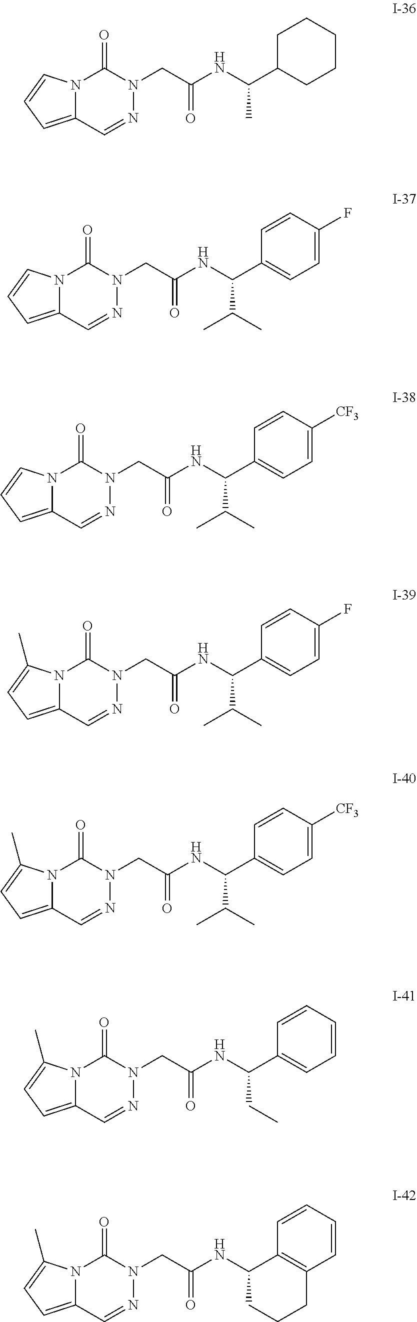

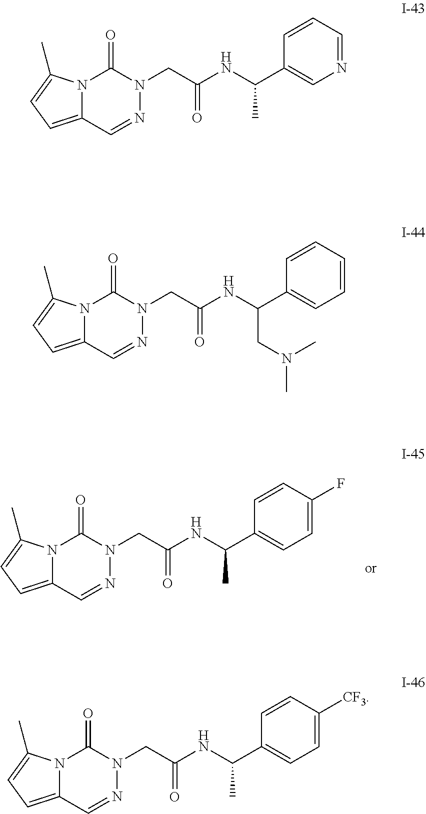 [SMILES: Cc1ccc2cnn(CC(=O)N[C@@H](C)c3cccnc3)c(=O)n12 ; Cc1ccc2cnn(CC(=O)NC(CN(C)C)c3ccccc3)c(=O)n12 ; Cc1ccc2cnn(CC(=O)N[C@H](C)c3ccc(F)cc3)c(=O)n12 ; "or" ; Cc1ccc2cnn(CC(=O)N[C@@H](C)c3ccc(C(F)(F)F)cc3)c(=O)n12]

10. A pharmaceutical composition, comprising the pyrrolotriazinone compound of formula I or the pharmaceutically acceptable salt thereof according to claim 1, and a pharmaceutical excipient.

11. A preparation method for the pyrrolotriazinone compound of formula I or the pharmaceutically acceptable salt thereof according to claim 1, comprising a following step: carrying out a condensation reaction between compound II and compound III;

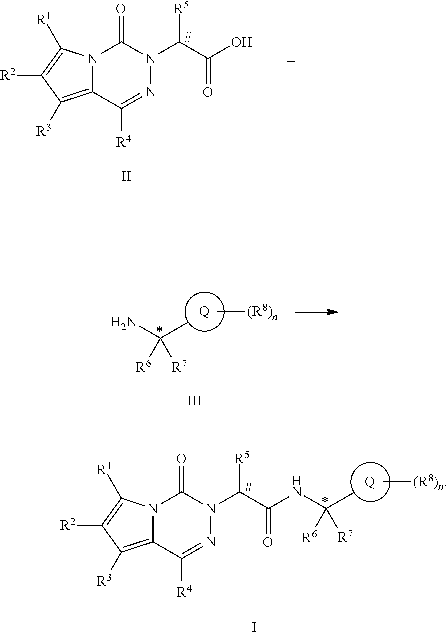

12. (canceled)

13. A method for treating schizophrenia, bipolar disorder, depression, cognitive disorder, autism spectrum disorder, sleep disorder, attention deficit hyperactivity disorder, post-traumatic stress disorder, substance abuse, drug addiction, eating disorder, obsessive-compulsive disorder, anxiety disorder, pain, or fibromyalgia in a subject in need thereof, comprising administering a therapeutically effective amount of the pyrrolotriazinone compound of formula I or the pharmaceutically acceptable salt thereof according to claim 1 to the subject.

14. A method for treating schizophrenia, bipolar disorder, depression, cognitive disorder, autism spectrum disorder, sleep disorder, attention deficit hyperactivity disorder, post-traumatic stress disorder, substance abuse, drug addiction, eating disorder, obsessive-compulsive disorder, anxiety disorder, pain, or fibromyalgia in a subject in need thereof, comprising administering a therapeutically effective amount of the pharmaceutical composition according to claim 10 to the subject.

15. The pyrrolotriazinone compound of formula I or the pharmaceutically acceptable salt thereof according to claim 7, wherein R⁷ is —CH₃, —CH₂CH₃, or

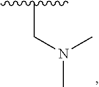 [SMILES: *CN(C)C]

16. The pyrrolotriazinone compound of formula I or the pharmaceutically acceptable salt thereof according to claim 6, wherein in Q, the 5- to 10-membered heteroaromatic ring is

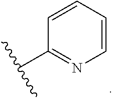 [SMILES: *c1ccccn1]

17. The pyrrolotriazinone compound of formula I or the pharmaceutically acceptable salt thereof according to claim 8, wherein 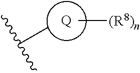
is

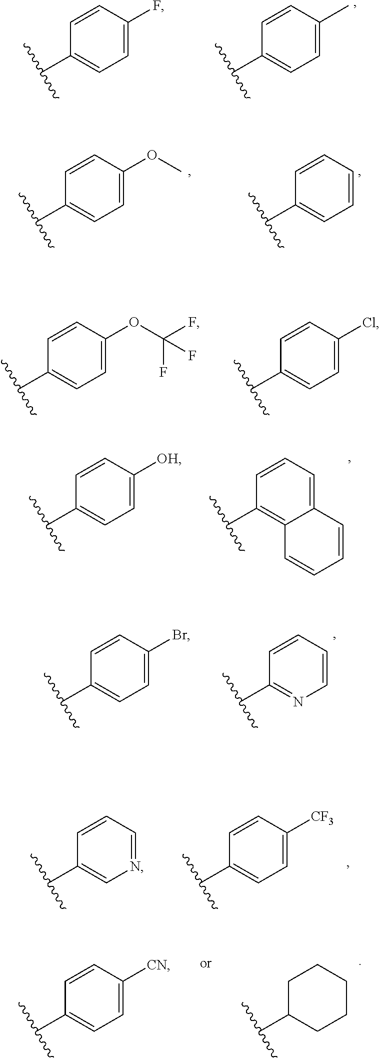

18. The pyrrolotriazinone compound of formula I or the pharmaceutically acceptable salt thereof according to claim 17, wherein 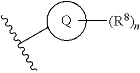
is

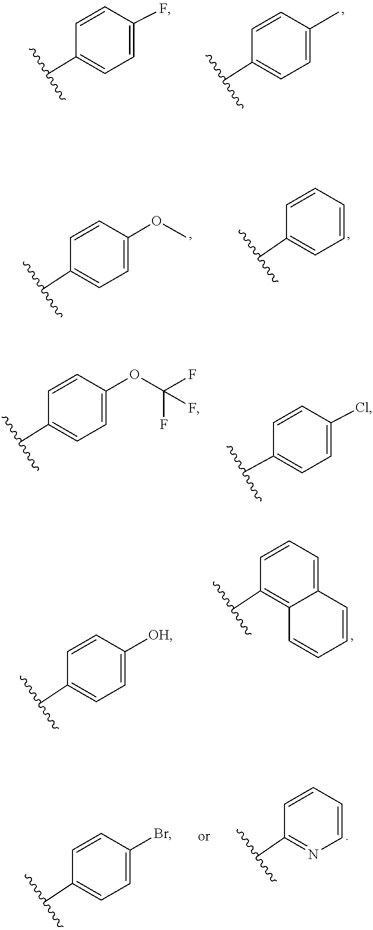

19. The pyrrolotriazinone compound of formula I or the pharmaceutically acceptable salt thereof according to claim 8, wherein 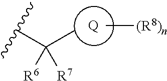
is

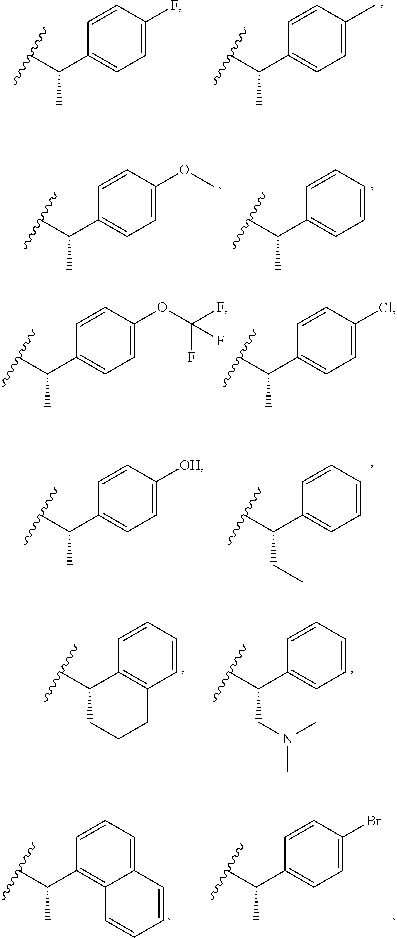

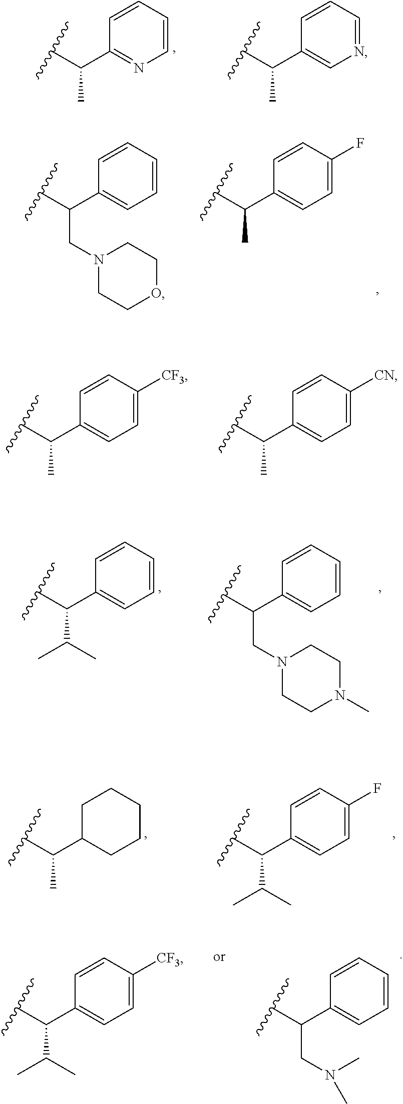

20. The pyrrolotriazinone compound of formula I or the pharmaceutically acceptable salt thereof according to claim 19, wherein 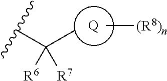
is [chemical structure]
